(19) United States
(12) Patent Application Publication
(10) Pub. No.: US 2007/0178220 A1
(43) Pub. Date: Aug. 2, 2007

(54) Materials and methods for manufacturing amorphous tricalcium phosphate and metal oxide alloys of amorphous tricalcium phosphate and methods of using the same

(76) Inventor: Robert L. Karlinsey, Indianapolis, IN (US)

Correspondence Address: SOMMER BARNARD ATTORNEYS,P.C., ONE INDIANA SQ, SUITE 3500, INDIANAPOLIS, IN, 46204, US

(21) Appl. No.: 11/701,210

(22) Filed: Jan. 31, 2007

Related U.S. Application Data
(60) Provisional application No. 60/763,607, filed on Jan. 31, 2006.

(51) Int. Cl. A61L 33/00; C09K 3/00; C04B 28/34

(52) U.S. Cl. 427/2.1; 106/35; 106/690; 106/691; 106/462

(57) ABSTRACT

Alloys of tricalcium phosphate and metal oxide such as TiO₂ and SiO₂ (ACP) are disclosed as well as method for making these alloys. Method for making these alloys include the steps of milling amorphous tricalcium phosphate and at least one metal oxide together in, for example, a ball mill operated under ambient conditions. Various aspects also relate to treating disease or damage to tissues such as enamel, dentine or bone by contacting tissue with these alloys. As well as formulations for oral care such as tooth pastes, mouth washes, tooth whiteners, and like that comprise ACP. Still another aspect is amorphous tricalcium phosphates that have antimicrobial activity. Various aspects disclosure methods for manufacturing these materials and for using them in various formulations and preparations such as dentifrices, mouth washes and rinses, teeth whiteners, gels, drenches, ointments, slaves, pastes, soaks, sprays, glues, cements, foodstuffs, and the like. Still another aspect is directed towards suing these compositions as parts of, or coatings for, devices such as needles, catheters, packagings, fillings, screws, pins, splints, implants, bandages, packings and the like.

### PRIORITY CLAIM

This Application claims the benefit of U.S. Provisional Patent Application Ser. No. 60/763,607, filed on Jan. 31, 2006, which is incorporated herein, by reference in its entirety.

### FIELD OF THE INVENTION

Various embodiments relate generally to materials such as amorphous tricalcium phosphate and alloys comprising amorphous tricalcium phosphates and metal oxides, methods of making these materials and methods of using these materials to treat tissue such as dentin, enamel, bone and the like.

### BACKGROUND

Preventing caries and improving the delivery of minerals necessary to healthy teeth and bone, while preserving and/or enhancing cosmetic features, are important goals in oral health care. In this regard the application of fluoride agents continues to be an effective and widely used treatment for the prevention of caries. Although effective in most cases, it is not without its risks. Negative side-effects associated with administering fluoride containing compounds to control caries include for example fluorosis. Although such complications are rare this can be serious and contribute to the continuing interest in developing additional compositions with enhanced anticaries activities and exhibit detrimental side-effects than formulation that include high levels of fluoride.

One alternative to conventional fluoride treatment is to reduce the amount of fluoride in oral preparations necessary to achieve acceptable protection against caries. In this regard, it may be useful to supplement fluoride containing preparations with other compounds that also contribute to caries prevention. For example, adding non-fluoridic components that enhance enamel remineralization to existing formulations may increase protection against caries even as fluoride levels in the compositions are reduced. In addition to the role remineralizing compositions may play in preventing caries such compounds may also be useful in reconstructive and cosmetic dentistry in that they may contribute to the addition of enamel or body mass to teeth and bones.

The importance of cosmetic and reconstructive dentistry in today's marketplace is reflected in the fact that many current advances in oral care relate primarily to cosmetic applications such as, for example, tooth whitening. Currently, cosmetic and reconstructive dentistry are centers of innovative therapeutic research.

Clearly then, there is a need for compounds that can help prevent caries and that can also aid in the reconstruction of damaged teeth and bones. There is also a continuing need for materials that exhibit antimicrobial, particularly antibacterial activities. Various embodiments discussed herein, address these varied needs.

### SUMMARY

One aspect is an alloy, comprising amorphous tricalcium phosphate and at least one metal oxide. In one embodiment the alloy has an average particle size in the range of about 5.0 microns to about 0.01 microns. In another embodiment the alloy has an average particle size in the range of about 1.2 microns to about 0.05 microns.

Still another embodiment is an alloy of amorphous tricalcium phosphate and at least one metal oxide is selected from the group consisting of TiO₂ and SiO₂.

In one embodiment the w amount of the amorphous tricalcium phosphate in the alloy is the range of about 99.5 to about 1.0 wt. % of the alloy and the amount of the metal oxide in the composition is in the range of about 0.5 to about 99.0% wt. % of the alloy.

In still another embodiment the amount of the amorphous tricalcium phosphate in the alloy is the range of about 99.5 to about 85 wt. % of the alloy and the amount of the metal oxide in the composition is in the range of about 0.5 to about 15% wt. % of the alloy.

Yet another embodiment is a method of manufacturing (forming) an alloy which includes amorphous tricalcium phosphate and at least one metal oxide. Various steps in the process include providing a portion of amorphous tricalcium phosphate, supplying a portion of at least one metal oxide, combining the two materials and pulverizing them to produce an alloy. In one embodiment the materials are pulverized using a ball mill, the ball mill can be operated at any speed or for any length of time in the presence of any inert anti-caking agent as may be necessary to produce an alloy with a desired set of physical, chemical or biological properties. In one embodiment a planetary ball mill is used and is operated at between about 250 to about 600 rpms, for between about 5 hours to about 5 days.

In still another embodiment the alloy is produced in the form of a powder and the average particle size of powdered alloy is in the range of about 5.0 microns to about 0.01 microns; in still another embodiment the average particle size of the powder in the alloy is in the range of about 1.2 microns to about 0.05 microns.

Still another aspect is a formulation for treating tissue. These formulations may include at least one alloy, in which the alloy includes amorphous tricalcium phosphate and at least one metal oxide. In one embodiment the alloy is in the form of a powder and the alloy in the powder has an average particle size in the range of about 5.0 microns to about 0.01 microns. In still another embodiment the alloy in the powder has an average particle size in the range of about 1.2 microns to about 0.05 microns.

In one embodiment the alloy in the formulation includes at least one metal oxide is selected from the group consisting of TiO₂ and SiO₂.

In still another embodiment the alloy in the formulation includes on the order of between about 99.5 to about 1.0 wt. % amorphous tricalcium phosphate and between about 0.5 to about 99.0% wt. % metal oxide. In yet another embodiment the range of amorphous tricalcium phosphate in the alloy is about 99.5 to about 85 wt. % of the alloy and the amount of the metal oxide in the alloy is in the range of about 0.5 to about 15% wt. % of the alloy.

In still another embodiment the formulation for treating tissue such as bone, dentin, enamel and the like further includes at least one of the following additives; fluoride, surfactants, antimicrobials, flavoring agents, detergents, coloring agents, buffering agents, thickening agents, cooling agents, glues, cements, and polishes.

One embodiment is a method of treating tissue, methods of treating tissue include applications, procedures, dosings and the like designed and/or intended to prevent disease or injury, or to repair disease or injury, or to reconstruction damage done to tissue by disease or injury, or reconstruct tissue such as bone, dentin and enamel for purely cosmetic purposes. One method includes supplying a formulation that includes at least one alloy in which the alloy includes amorphous tricalcium phosphate and at least one metal oxide. In various treatments addition steps include contacting the formulation with at least one surface of a tissue.

In still another embodiment in addition to either or both amorphous tricalcium phosphate or an alloy of amorphous tricalcium phosphate and at least one metal oxide a formulation for treating tissue further includes at one compounds selected from the following groups of compounds, agents and the like; a form of bioavailable fluoride, surfactants, flavoring agents, coloring agents, thickening agents, antimicrobial agents, buffering agents, and gums.

Still another embodiment is a foodstuff, which includes at least one alloy, the alloy comprising amorphous tricalcium phosphate and at least one metal, the foodstuff may also be formulated to include at least one compound selected from the following class of compounds: sources of fluoride, gums, flavoring agents, coloring agents, cooling agents, surfactant, buffers, antimicrobial agents, and stabilizers.

One embodiment is a form of amorphous tricalcium phosphate that exhibits antimicrobial activity in one such embodiment this material is manufactured in the form of a powder using solid state techniques. In one embodiment the powder has an average particle size of about 1.5 microns or less.

Still another embodiment is a method of manufacturing an antimicrobial composition, pulverizing tricalcium phosphate until it is amorphous and has a particle size on the order the particle size of amorphous tricalcium phosphate manufactured by pulverizing about tricalcium phosphate in a PM 100 planetary ball mill, the mill having a stainless steel vessel with a volume of about 150 ml, said vessel including 25 stainless steel balls, wherein each of the balls has a diameter of about 10 mm, and about 2 ml of ethanol, said ball mill is operated at about 450 rpms for about 5 days.

Yet another embodiment is a method for controlling microbes. Forms of control include killing microbes including bacteria, fungi, molds, virus and the like, or inhibiting of slowing the growth of the same. In one embodiment a method for controlling microbes includes contact the microbes or surfaces that microbes have or will or are like to contact with amorphous tricalcium phosphate. In one embodiment amorphous tricalcium phosphate that exhibits antimicrobial activity has a particle size on the order of the particle size of amorphous tricalcium phosphate manufactured by pulverizing tricalcium phosphate in a PM 100 planetary ball mill, the mill having a stainless steel vessel with a volume of about 150 ml, said vessel including 25 stainless steel balls, wherein each of the balls has a diameter of about 10 mm, and about 2 ml of ethanol, and said mill is operated at about 450 rpms for about 5 days.

Another embodiment includes spraying, adding, coating, painting, dipping, drenching surfaces or microbes with formulations that include amorphous tricalcium phosphates that exhibit antimicrobial activity. Various device that can be contacted with the material include, but are not limited to, bandages, screws, fillings, straps, periodontal or surgical packings, nails, splints, implants, catheters, prosthetic devices, stents, needles, lances, surgical tools, meshes, sutures, and endoscopes.

Still another embodiment includes adding amorphous tricalcium phosphates which exhibit antimicrobial activity to various formulations including, but not limited to: dentifrices, mouth washes, rinses and sprays, tooth whiteners, ointments, salves, foodstuffs, glues, cements and disinfectants.

One embodiment is an alloy of amorphous tricalcium phosphate and at least one metal oxide. In one embodiment the metal oxide is selected from the group consisting of TiO₂ and SiO₂. In one embodiment the alloy includes between about 1 to about 99.5 wt. % of tricalcium phosphate and between about 99 to about 0.5 wt. % of metal oxide.

One embodiment is an alloy, useful for preventing caries or treating diseased or damaged teeth or bone, comprising amorphous tricalcium phosphate and at least one metal oxide. In one embodiment the composition includes amorphous tricalcium phosphate alloyed with at least one metal oxide such as titanium oxide TiO₂ or SiO₂ or the like.

One embodiment is an alloy of amorphous tricalcium phosphate and at least one metal oxide such that the level of amorphous tricalcium phosphate in the alloy is in the range of 1.0 wt. % to about 99.5 wt. % of the total weight of the alloy, and the range of at least one metal oxide in the alloy is in the range of about 99 to about 0.5 wt. % of the total weight of the alloy.

Another embodiment is an alloy comprising amorphous tricalcium phosphate and at least one metal oxide alloy, the level of amorphous tricalcium phosphate in the alloy is between about 99.0 to about 90 wt. % of the total weight of the alloy and the level of metal oxide in the alloy is about 1.0 to about 10.0 wt. % of the total weight of the alloy.

Yet another embodiment is an alloy comprising amorphous tricalcium phosphate alloyed with at least one metal oxide such as SiO₂, TiO₂ or the like. In one embodiment the level of amorphous tricalcium phosphate in the alloy is on the order of ratio of about 95 wt. % to about 5 wt. %, the remainder of the alloy is substantially comprised of metal oxide.

Still another embodiment is a composition useful for treating tissues such as enamel, dentin or bone comprising amorphous tricalcium phosphate alloyed with at least one metal oxide such as TiO₂ or SiO₂, or the like, and at least one additional component such as a surfactant, an antimicrobial agent, a flavoring compound, a cooling agent, a thickening agent, a biocompatible buffer or a bioactive form of fluoride.

Yet another embodiment is a method of manufacturing an alloy of amorphous tricalcium phosphate and at least one metal oxide. In one embodiment the alloy is manufactured by mixing tricalcium phosphate with at least one metal oxide and milling the compounds until the alloy forms. In one embodiment, milling is carried out using a ball mill. Milling is carried out under ambient conditions between about 250 to about 550 rpms, for between about 10 hours to about 5 days.

Still another embodiment is using alloys comprising amorphous tricalcium phosphate and metal alloys to prevent, treat or reconstruct damage done to tissues such as bone, dentin, or enamel.

Another embodiment is using amorphous tricalcium phosphate and metal alloys to coat surfaces of medical or dental implants, devices, tools, prosthetics, and the like.

One embodiment, is the method of manufacturing amorphous tricalcium phosphate which comprises the steps of: combining about equal amounts of carbonate (CaCO₃) and calcium phosphate dehydrate (CaHPO₄)₂.2H₂O); heating the mixture to about 1050° C. and holding the mixture at this temperature for about 24 hours; and milling the resultant material tricalcium phosphate until it is amorphous (i.e. lacking significant long range order as can be determined using powder IR spectrometry or x-ray diffraction).

Still another embodiment is using amorphous tricalcium phosphate as an antimicrobial agent.

Yet another embodiment is a method of using amorphous tricalcium phosphate to inhibit the growth of microorganisms, comprising the steps of providing the amorphous tricalcium phosphate and contacting it with a surface. In one embodiment the surface is tissue such a bone, dentin, or enamel. In still another embodiment the surface is soft tissue. And in still another embodiment, the surface is a prosthetic device, a medical or dental tool, or a medical or dental device such as a filling, glue, cement, screw, level, band, strap, bandage or surgical packing.

In one embodiment an alloy of amorphous tricalcium phosphate and at least one metal oxide, for example TiO₂, SiO₂, or the like is formed by pulverizing a mixture including these materials in a PM 100 planetary ball mill at about 450 rpms for between about 10 hours to about 5 days. In one embodiment milling is carried out in the presence of a relative volatile liquid such as ethanol or pentane to prevent caking.

Still another embodiment is a method of preventing dental caries or preventing or repairing damage done to teeth or bone comprising the steps of: providing a remineralizing composition, which includes an ACP material and a metal oxide; and contacting the composition with a surface of at least one tooth or bone. In one embodiment the metal oxide material is titanium oxide (TiO₂). In still another embodiment the metal oxide is SiO₂. In one embodiment the formulation further includes a safe and effective amount of bioavailable fluoride for example between about 200 and about 5,000 ppm fluoride.

Another embodiment is a dentifrice comprising a safe and effective amount of an alloy of amorphous tricalcium phosphate and a safe metal oxide such as SiO₂, TiO₂, or the like. In one embodiment the dentifrice is selected from the group consisting of tooth pastes, tooth powders, oral rinses, and the like. In one embodiment the dentifrice includes at least one additive selected from the group consisting of surfactants, thickening agents, flavorings, cooling agents, antimicrobials, or activated fluoride compounds.

Yet another embodiment is a mouth wash or mouth rinse, comprising a remineralizing composition which includes a safe and effective amount of amorphous tricalcium phosphate and a safe and effective amount of a metal oxide such as TiO₂. In one embodiment the mouth rinse or wash includes at least one additive selected from the group consisting of surfactants, thickening agents, flavorings, cooling agents, antimicrobials, or activated fluoride compounds.

Still another embodiment is a tooth whitening agent, comprising a remineralizing composition which includes a safe and effective amount of an alloy of amorphous tricalcium phosphate and metal oxide such as SiO₂, TiO₂, or the like. In one embodiment the whitening agent is selected from the group consisting of tooth gels, pastes, rinses, soaks, strips and the like. In one embodiment the whitening agent includes at least one additional component selected from the group consisting of surfactants, huecants, bleaches, thickening agents, flavorings, cooling agents, antimicrobials or activated fluoride compounds.

Another embodiment is a foodstuff, comprising a remineralizing composition which includes a safe and effective amount of amorphous tricalcium phosphate and a safe and effective amount of a metal oxide such as TiO₂, SiO₂, or the like. In one embodiment the foodstuff is selected from the group consisting of gums, confectioneries, beverages, and lozenges.

Still another embodiment is an antimicrobial material comprising amorphous tricalcium phosphate. ACP with antimicrobial activity can be formed by milling ACP until it has an average particle size similar to the size obtained by milling ACP in a PM 100 planetary ball mill, or similar device operated at about 450 rpm for between about 10 to about 120 hours. In one embodiment ACP is milled in the presence of a material that has a viscosity lower than water, such materials include, for example, the liquid ethanol.

Yet another embodiment includes forming compounds such as amorphous tricalcium phosphate and or alloys of amorphous tricalcium phosphate and metal oxides such as TiO₂ with varying particle sizes, it being understood that materials with different particle sizes may exhibit differing physical, chemical, and biological properties.

In one embodiment the size of the particles in the alloy is formed by adjusting the process conditions under which the alloy is created. In one embodiment, the average size of the particles can be adjusted by adjusting milling parameters, including RPM, temperature, length of mill time, and the types of carriers added to the milling step; including materials such as water, ethanol, methanol, and the like. In still another embodiment, the size of the particles is adjusted by varying the type of equipment and/or process used to manufacture the material.

In still another embodiment, amorphous tricalcium phosphate formulations exhibiting antimicrobial activity are used in various devices that contact bodily tissues and blood including, for example, bandages, dental wraps and packing, bone implants, sutures, staples, glues, catheters, needles, surgical devices, endoscopes, fibers, films, meshes, resins, implants, solutions, sprays, powders and the like.

In yet another embodiment, amorphous tricalcium phosphate exhibiting antimicrobial activity are applied to and/or incorporated into the surfaces of devices which may be involved in the spread of bacteria. The material can be applied by any method commonly used such as coating, dusting, spraying, embedding, painting, soaking, drenching, and the like.

### BRIEF DESCRIPTION OF THE FIGURES

FIG. 1. Table 1, changes in VHN values measured after exposing samples of a bovine enamel to a dentifrice solution that includes substantially water.

FIG. 2. Table 2, changes in VHN values measured after exposing samples of bovine enamel to a dentifrice solution that includes CaCl₂ and K₂H₂PO₄.

FIG. 3. Table 3, changes in VHN values measured after exposing samples of a bovine enamel to a dentifrice solution that includes ACP100(No TiO₂).

FIG. 4. Table 4, changes in VHN values measured after exposing samples of a bovine enamel to a dentifrice solution that includes ACP95(5.0 wt. % TiO₂).

FIG. 5. Table 5, changes in VHN values measured after exposing samples of a bovine enamel to a dentifrice solution that includes ACP90(10 wt. % TiO₂).

FIG. 6. Table 6, changes in VHN values measured after exposing samples of bovine enamel to a dentifrice solution that includes ACP85(15 wt. % TiO₂).

FIG. 7. Table 6, compilation of mean changes in VHN reported in tables 1-6 (FIGS. 1-6)

FIG. 8. Graph of change in VHN values from Tables 1-6 in FIGS. 1-6, each group represents a different set of conditions to which enameled surfaces were exposed before measuring the change in their surface hardness.

FIG. 9. Table 7, summary of data collected from studying the effect of various calcium-phosphate treatments on surfaces undergoing remin/demin cycling.

FIG. 10. Table 8, summary of data collected from studying the effect of various calcium-phosphate treatment groups on surfaces undergoing remin/demin cycling; these data were compiled in the presence of artificial saliva.

FIG. 11. Table 9, summary of data collected from studying the effect of various calcium-phosphate treatment groups on surfaces undergoing remin/demin cycling; these data were compiled using pooled human saliva.

FIG. 12. Mean values of microhardness (VHN) enhancement measured for enamel surfaces treated with compositions comprising alloys of amorphous tricalcium phosphate and TiO₂. Although not shown, error bars similar to those expressed in the corresponding tables could be plotted for each data point in the graph.

FIG. 13. Table 10, a summary of data collected from studying the effect of various calcium-phosphate treatment groups on surfaces undergoing remin/demin cycling; these data include data collected using chewing gum formations that either include sugar or do not include sugar.

FIG. 14. Table 11, summary of data illustrating that alloys of amorphous tricalcium has antimicrobial properties.

FIG. 15. Plot of the level of water soluble calcium in 50 mg sample of ACP; ACP was prepared by milling for 1, 3 or 7 days before the level of soluble calcium was measured.

FIG. 16. Plot of the amount of water soluble calcium in 10 mg of various preparations of ACPS, ACPS comprising the following levels of SiO₂ were analyzed, 0.0, 5.0, 10.0, 25.0, 50, 75, and 90 wt. %, respectively. The line is a ternary (3^{rd} order polynomial) fit of the data, it indicates 3 distinct regions.

FIG. 17. Plot of the level of water soluble calcium in ACP metal oxide alloys measured for two sample sizes 10 and 50 mg. The following materials: were made during 1 day of milling, ACPS (10 wt. % SiO₂) or amorphous tricalcium phosphate alloyed with 10 wt. % TiO₂. These data indicate that both materials have about the same amount of water soluble calcium.

FIG. 18. Plot of the level of water soluble calcium in amorphous tricalcium phosphate alloyed with one of the following levels of TiO₂, 0, 5 or 10 wt. %. Values were measured for both 10 and 50 mg of sample.

FIG. 19. IR absorbance spectra of alloys that include amorphous tricalcium phosphate and the following levels of SiO₂: 0, 5, 90, 75, 50, 75, 90 wt. %, the remainder of these samples was TCP, as a control one sample comprised of 100 wt. % SiO₂ was also run. The asterisks indicate characteristics peaks associated with CaO moieties.

FIG. 20. Schematic diagram showing proposed structure of an amorphous P₂O₅ network.

FIG. 21. Table summarizing the principal covalent and ionic P-O vibrational bands obtained by deconvolution of the spectra reported in FIG. 19 between 700 and 1300 cm⁻¹.

FIG. 22. A graphical comparison of the deconvoluted peak centers for P-O vibrations listed in the table presented in FIG. 21 based on some of the spectra presented in FIG. 19.

FIG. 23. Schematic diagram showing proposed mechanisms of P₂O₅ network modification.

FIG. 24. The mass in mg of water soluble calcium measured for the following materials: amorphous tricalcium phosphate alloyed with various levels of SiO₂, the level of SiO₂ in the materials measured is as follows: 0, 5, 10, 25, 50, 75, or 90 wt. %, the remainder of each material is made up of TCP. Soluble calcium was measured for samples of 10, and 50 mg of material, the lines are isotherms.

FIG. 25. A plot of bioavailable fluoride measured after contacting a fluoride solution for 15 days at 22° C., plotted as a function of wt. % (0.0, 0.05, 0.1, 0.2) of the material in 25 ml of NaF_{(aq)}. Data were collected for the following materials: CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂. The fits are fluoride stability isotherms, no surfactants were added in this example.

FIG. 26. A plot of bioavailable fluoride measured after contacting a fluoride solution for 15 days at 22° C. in the absence of surfactants, plotted as a function of wt. % (0.0, 0.05, 0.1, 0.2 respectively) of the material in 25 ml of NaF_{(aq)}. Data were collected for the following materials: CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂: 10% TCP, 90% SiO₂. The plots are fluoride stability isotherms.

FIG. 27. A plot of bioavailable fluoride measured after contacting a fluoride solution for 7 days at 22° C. in the presence of 1.0 wt. % 600 Da PEG, plotted as a function of wt. % (0.0, 0.05, 0.1, 0.2 respectively) of the material in 25 ml of NaF_{(aq)}. Data were collected for the following materials: CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂. The plots are fluoride stability isotherms.

FIG. 28. A plot of bioavailable fluoride measured after contacting a fluoride solution for 7 days at 22° C. in the presence of 0.5 wt. % SLS, plotted as a function of wt. % (0.0, 0.05, 0.1, 0.2 respectively) of the material in 25 ml of NaF_{(aq)}. Data were collected for the following materials: CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂. The plots are fluoride stability isotherms.

FIG. 29. A plot of bioavailable fluoride measured after contacting a fluoride solution for 7 days at 22° C. in the presence of 0.5 wt. % CPC, plotted as a function of wt. % (0.0, 0.05, 0.1, 0.2 respectively) of the material in 25 ml of NaF_{(aq)}. Data were collected for the following materials: CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂. The plots are fluoride stability isotherms.

FIG. 30. Photograph depicting the color change associated with CPC and test formulations in 25 ml NaF(aq) with 0.5 wt. % CPC at 7 days, 22° C. The labels represent the following formulations: A, CPC+NaF_{(aq)} control; B, CPC+12.5 mg CaCl₂+NaF_{(aq)}; C, CPC+12.5 mg 100% TCP+NaF_{(aq)}; D, CPC+12.5 mg 90% TCP, 10% SiO2+NaF_{(aq)}; E, CPC+12.5 mg 50% TCP, 50% SiO₂+NaF(aq).

FIG. 31. A plot of bioavailable fluoride measured after contacting test materials with 25 ml slurry that included 12.5 g Aquafresh Extreme Clean™ for 289 days at 22° C. plotted as a function of wt. % ACPS added to the dentifrice slurry. Data were collected for the following test materials: 100 wt. % TCP 0 wt. % CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂.

FIG. 32 A plot of bioavailable fluoride measured after contacting test materials with 25 ml slurry that included 12.5 g Aquafresh Cavity Protection™ for 289 days at 22° C. plotted as a function of wt. % ACPS added to the dentifrice slurry. Data were collected for the following test materials: 100 wt. % TCP 0 wt. % SiO₂; CaCl₂, 100% TCP, 0% SiO₂; 95% TCP, 5% SiO₂; 90% TCP, 10% SiO₂; 85% TCP, 15% SiO₂, 75% TCP, 25% SiO₂; 50% TCP, 50% SiO₂; 25% TCP, 75% SiO₂; 10% TCP, 90% SiO₂.

FIG. 33. Histograms illustrating the amount of Enamel Fluoride Uptake (ppm) measured in the presence of the following test materials: water; NaF; ACP90+NaF; ACP90+PEG+NaF; ACP90+SLS+NaF; and ACP90+CPC+NaF.

FIG. 34. Histograms illustrating the amount of Enamel Fluoride Uptake (ppm) measured in the presence of the following test materials: water; NaF; ACP+CPC+NaF; ACP90+CPC+NaF; and ACP50+CPC+NaF.

FIG. 35. Histograms illustrating the amount of Enamel Fluoride Uptake (ppm) measured in the presence of the following test materials: water; NaF; ACP90+CPC+NaF; ACP50+CPC+NaF; ACP90+SLS+NaF; and ACP50+SLS+NaF.

FIG. 36. Histograms illustrating the change in enamel surface acid-etch depths measured in the presence of the following materials: water; NaF; ACP90+Naf; ACP90+PEG+NaF; ACP90+SLS+NaF; and ACP90+CPC+NaF. Larger negative values indicate better enamel resistance to acid attack.

FIG. 37. Histograms illustrating the change in enamel surface acid-etch depths measured in the presence of the following materials: water; NaF; ACP+CPC+Naf; ACP90+CPC+NaF; and ACP50+CPC+NaF. Larger negative values indicate better enamel resistance to acid attack.

FIG. 38. Histograms illustrating the change in enamel surface acid-etch depths measured in the presence of the following materials: water; NaF; ACP90+CPC+NaF; ACP50+CPC+NaF; ACP90+SLS+NaF; and ACP50+SLS+NaF.

FIG. 39. Histograms illustrating the change in Vickers microhardness resulting from a 6-day remin/demin pH cycling study carried out in vitro on bovine enamel. The results are presented as a function of test materials added to the cycling run. Test materials used in the example are as follows: water; 1100 ppm F; ACP100+1100 ppm F; ACP75+1100 ppm F; ACP50+1100 ppm F; ACP25+1100 ppm F; and ACP10+1100 ppm F.

FIG. 40. Histograms illustrating the change in Vickers microhardness resulting from a 6-day remin/demin pH cycling study carried out in vitro on bovine enamel. The results are presented as a function of test materials added to the cycling run. Test materials used in the example are as follows: water; 1100 ppm F; ACP90+1100 ppm F; and ACP50+1100 ppm F.

FIG. 41. A trace generated using a Nanopac 151 particle analyzer manufactured by Microtrac™. All samples were prepared and analyzed in conformity with the manufacturer's instructions. The material analyzed was un-milled tricalcium phosphate.

FIG. 42. A trace generated using a Nanopac 151 particle analyzer manufactured by Microtrac™. All samples were prepared and analyzed in conformity with the manufacturer's instructions. The material analyzed, amorphous tricalcium phosphate, was prepared by milling tricalcium phosphate for 24 hours at 350 rpm in the presence of about 5 ml of ethanol added to prevent caking, in a 150 ml stainless steel vessel which also included 20 stainless steel balls. Each ball had a diameter of 10 mm. The mill was operated at 350 rpm for 24 hours under ambient conditions.

FIG. 43. A trace generated using a Nanopac 151 particle analyzer manufactured by Microtrac™. All samples were prepared and analyzed in conformity with the manufacturer's instructions. The material analyzed was an alloy of amorphous tricalcium phosphate and TiO₂. The alloys were prepared by milling a total of about 20 g of a mixture of 95 wt. % tricalcium phosphate and about 5 wt. % TiO2. The material, along with about 5 ml of ethanol, was added to a 150 ml stainless steel vessel along with 20 stainless steel balls, each ball having a diameter of 10 mm. The vessel was sealed and the contents were milled for 24 hours at 350 rpm under ambient conditions.

FIG. 44. Is a trace generated using a Nanopac 151 particle analyzer manufactured by Microtrac™. All samples were prepared and analyzed in conformity with the manufacturer's instructions. The material analyzed here was an alloy comprising 90 wt. % amorphous tricalcium phosphate and 10 wt. % titanium oxide. The material was prepared by using a ball mill. Briefly, a 150 ml stainless steel vessel was loaded with 20 balls each having a diameter of about 10 ml, about 20 grams of total material (90 wt. % tricalcium phosphate and 10 wt. % TiO₂). The vessel was closed, placed in the mill and the mill was operated for 24 hours at 350 rpm under ambient conditions.

### DESCRIPTION

For the purposes of promoting an understanding of the principles of the invention, reference will now be made to the preferred embodiments thereof, and specific language will be used to describe the same. It will nevertheless be understood that no limitation of the scope of the invention is thereby intended, such alterations, modifications, and further applications of the principles of the invention being contemplated as would normally occur to one skilled in the art to which the invention relates.

A number of explanations and experiments are provided by way of explanation and not limitation. No theory of how the invention operates is to be considered limiting whether proffered by virtue of description, comparison, explanation or example.

Most terms are given their usual and customary meaning as used in the art to which the various embodiments are directed. Some terms are clarified as follows. As used herein the terms “pharmaceutically-acceptable topical oral carrier,” or “topical, oral carrier,” generally means one or more compatible solid or liquid fillers, diluents or encapsulating substances that are suitable for topical, oral administration. The term, “compatible,” as used herein, means that components of the composition are capable of being commingled without interacting in a manner which would substantially reduce the composition's stability and/or efficacy for treating or preventing oral care conditions such as caries, according to the compositions and methods of the present invention.

The term “about” generally refers to range of plus or minus on the order of ten percent of the value; the entire range being on the order of 20 percent of the relevant value.

The acronym “TCP” as used herein often refers to tricalcium phosphate, in many of the figures alloys of amorphous tricalcium phosphate and metal oxides such as TiO₂ or SiO₂ are referred to as wt. % TCP, wt % TiO₂ or wt % SiO₂ this nomenclature was used because the alloys were likely prepared by milling mixtures of tricalcium phosphate (TCP) with a metal oxide convert TCP into amorphous tricalcium phosphate in the same vessel in which the alloy of amorphous tricalcium phosphate and metal oxide was formed.

Clearly there is a need for new approaches to preventing caries as well as for strengthening teeth and bone. One of the best avenues for meeting these goals is to enhance the remineralization of boney and enameled surfaces. For example, remineralizing of the enamel surfaces of teeth is one practical therapeutic measure known to help prevent caries. While many treatments are, and have been, used to thwart the development and progression of dental caries, there is still a need for additional clinically effective remineralizing agents that can help to protect teeth in the presence of an unrelenting cariogenic/erosive challenge.

Cross-fertilization between medical applications, such as between dentistry, and nanotechnology is a relatively new field; a field that is yet to be fully exploited. This is especially true in the fields of preventive and cosmetic dentistry. In part, by exploiting the properties of various materials on the nanoscale, we have developed compounds and methodologies for promoting the remineralization of teeth and bone. Various embodiments include compositions and methodologies for producing and using compositions that enhance the process of remineralizing teeth and bone. Various embodiments provide improved methods for preventing dental caries and for treating defects in both teeth and bones.

One embodiment provides remineralizing nanocomposites that exhibit enhanced mineral delivery when a component of teeth and bone is interfaced with a nanomaterial.

Various embodiments include amorphous tricalcium phosphate (ACP). ACP is a relatively soluble precursor to hydroxyapatite. In one embodiment, ACP is combined with a ceramic, such as titania (TiO₂), which appears to increase the ability of the ACP to interact with the surfaces of both tooth and bone. In various embodiments, the metal oxide alloyed with tricalcium phosphate and appears to stabilize tricalcium phosphate in solution (or suspension). This presence of an alloy of tricalcium phosphate and at least one metal oxide also improves ion dynamics in solid polymer electrolytes, such as polyethylene-oxide titania with a lithium salt (CF₃SO₄).

In one embodiment, a solid state transformation of tricalcium phosphate and at least one metal oxide such as titania is effectuated by mechanical alloying (MA) ACP and titania. MA may effectuate solid-state amorphization transformations through processes such as fracturing and the cold welding of particles. In many applications, MA is an excellent alternative to high temperature blending or solution chemistry. In some applications, it provides a very effective way to maximize the interface of a nanomaterial within a matrix with other components of the matrix. In one embodiment, MA is used to prepare amorphous tricalcium phosphate for use in a composition that promotes remineralization of teeth and bones.

Various results reported below indicate that mechanically alloying ACP with various nanoparticles promotes effective interfacing between these materials. The result of this interaction is a stable mineral system in which the interaction between calcium and phosphate is minimized. This increases the availability of these compounds for uptake into oral/body fluids. In one embodiment, ACP remains stabilized when interfaced with a nanopowder, such as titania, thereby inhibiting or at least reducing the formation of calcium-phosphorous complexes. Increasing the levels of these materials in pellicle and plaque fluids present in the oral cavity helps to promote the remineralization of teeth. The increased enamel remineralization observed may be due to an increase in the amount of ACP delivered to the enamel. This in turn may be due to a surface effect between ACP and titania realized only at the nanoscale.

Similarly, solution stabilization of ACP has been attributed to the milk protein casein phosphoprotein (CPP) in the CPP-ACP system (Recaldent). In the Recaldent system, a phosphoprotein appears to stabilize amorphous tricalcium phosphate in plaque fluid, thereby encouraging controlled mineral release for uptake by tooth enamel. The CPP-ACP complex is naturally found in, for example, cheese and milk. While this is an effective means for delivering ACP to teeth, it includes the use of an antigenic compound that may not be suitable for people who are sensitive to milk products. A fragment of the carrier protein has also been adapted for use in the delivery of ACP to the oral cavity. For a more detailed discussion of this system, the reader is directed to see the following U.S. patents, publications, and references: U.S. Publication No. 2003/0152525A1, published on Aug. 14, 2003 to Dixon et al.; U.S. Pat. Nos. 5,993,786 and 5,833,954 to Chow et al., all of which are incorporated herein by reference.

ACP ceramic compositions, of various embodiments, offer the added benefit of providing an enhanced mineral delivery system that does not include the use of a milk based compound. Accordingly, the ACP ceramic composition may be suitable for use by people that are intolerant of the CPP-ACP system or that are reluctant to use animal based products or bioengineered peptides and compositions including the same.

Infection is an ongoing concern in all aspects of human health ranging from water quality to the safe production, storage and transportation of food to virtually any dental or medical intervention. Various inorganic materials have been used to help control microbes that cause contamination, including for example: silver ions and silver ions in complex with zeolite, see for example, U.S. Pat. No. 6267,560 B1 issued on Jul. 31, 2001 and U.S. Pat. No. 6,582,715 issued on Jun. 24, 2003 both to Barry, et al., and both of which are incorporated herein by reference in their entireties. Similarly, various organic molecules have been used to control bacterial growth. For a further discussion of these materials and their application, the reader is directed to U.S. Pat. No. 6,585,989 B2 issued on Jul. 1, 2003 to Herbst, et al., which is incorporated herein by reference in its entirety.

Various embodiments of the invention are drawn to tricalcium phosphate, which is processed into amorphous tricalcium phosphate, which exhibits good antimicrobial activity. Various uses for this material include applying to surfaces that contact sources of bacterial contamination or incorporating into materials are likely to contact materials that are in contact with microorganisms.

The carriers of the present invention may include the usual and conventional components of toothpastes (including gels and gels for subgingival application), mouth rinses, mouth sprays, and more. Many of these are more fully described hereinafter.

The choice of a carrier to be used is generally determined by the way the composition is to be introduced into the oral cavity. If a tooth paste (including tooth gels, etc.) is to be used, then a “toothpaste carrier” is chosen and may include, for example, abrasive materials, sudsing agents, binders, humectants, flavoring and sweetening agents and the like as disclosed in, for example, U.S. Pat. No. 3,988,433, to Benedict, issued on Oct. 25, 1976, which is incorporated herein by reference. If a mouth rinse is to be used, then a “mouth rinse carrier” is chosen, such as water, flavoring and sweetening agents as disclosed in, for example, U.S. Pat. No. 3,988,433 issued to Benedict, and incorporated herein by reference in its entirety. Similarly, if a mouth spray is to be used, then a “mouth spray carrier” is chosen. If a sachet is to be used, then a “sachet carrier” is chosen (e.g., sachet bag, flavoring and sweetening agents). If a subgingival gel is to be used (for delivery of the active material into the periodontal pockets, or around the periodontal pockets), then the material may be combined with a “subgingival gel carrier”. Suitable subgingival carries include those disclosed in U.S. Pat. No. 5,198,220, Damani, issued Mar. 30, 1993, P&G, U.S. Pat. No. 5,242,910, Damani, issued Sep. 7, 1993, all of which are incorporated herein by reference in their entirety. Carriers suitable for the preparation of compositions of the present invention are well known in the art. Their selection will depend on secondary considerations such as mouth feel, taste, cost, shelf stability and the like.

Preferred compositions for use in various embodiments may be in the form of dentifrices, such as toothpastes, tooth gels, tooth polishes and tooth powders. Components of such toothpaste and tooth gels generally include one or more of a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an additional anticaries agent (from about 0.05% to about 10% additional anticaries agent), and an anticalculus agent (from about 0.1% to about 13%). Tooth powders, of course, contain substantially all non-liquid components.

Other preferred compositions for use in various embodiments include, for example, non-abrasive gels, including subgingival gels. Gel compositions commonly include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), water (from about 2% to about 45%), and may comprise an additional anticaries agent (from about 0.05% to about 10% of additional anticaries agent), and an anticalculus agent (from about 0.1% to about 13%).

Other preferred compositions for use in various embodiments may include, for example, mouthwashes, mouth rinses, and mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more additional anticaries agents present, for example, from about 0.05% to about 1.0% of additional anticaries agent, and an anticalculus agent present, for example, from about 0.1% to about 13%.

Other preferred compositions for use with various embodiments include, for example, dental solutions. Components of such dental solutions generally may include one or more of water present from about 90% to about 99%, preservative present from about 0.01% to about 0.5%, thickening agent present from 0% to about 5%, flavoring agent present from about 0.04% to about 2%, sweetening agent present from about 0.1% to about 3%, and surfactant present in such compositions from about 0% to about 5%.

Types of carriers which may be included in compositions of the various embodiments include, but are not limited to, abrasives, sudsing agents, surfactants, thickening agents, humectants, flavoring and sweetening agents, anticalculus agents, alkali metal bicarbonate salts, and other buffers sources of fluoride.

Dental abrasives useful in the topical, oral carriers of the compositions of various embodiments include many different materials. Various suitable materials are preferably materials that are compatible within the composition of interest and one that does not excessively abrade dentin. Suitable abrasive materials include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in various embodiments include, for example, particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Various mixtures of various abrasives may also be used.

Silica dental abrasives of various types may be used in some embodiments because they provide exceptional dental cleaning and polishing performance without unduly abrading tooth enamel or dentine. The silica abrasive polishing materials described herein, as well as other abrasives, generally have an average particle size ranging between about 0.1 to about 30 microns, and preferably from about 5 to about 15 microns, although materials with differing sizes may also be used in various embodiments. The abrasive can be precipitated silica or silica gels such as the silica xerogels described in U.S. Pat. No. 3,538,230 issued to Pader et al., on Mar. 2, 1970, and, U.S. Pat. No. 3,862,307, issued to DiGiulio on Jan. 21, 1975, both of which incorporated herein by reference in their entirety. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, Zeodent®, particularly the silica carrying the designation Zeodent 119®. For a more thorough discussion and listing of types of silica dental abrasives useful in the toothpastes, the reader is directed to see, U.S. Pat. No. 4,340,583, issued to Wason on Jul. 29, 1982, which is incorporated herein by reference in its entirety. The abrasive in the toothpaste compositions described herein is generally present at a level of from about 6% to about 70% by weight of the composition. Preferably, toothpastes may contain from about 10% to about 50% of abrasive, by weight of the composition.

One useful precipitated silica, for use in various embodiments, is disclosed in U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, all of which incorporated herein by reference in their entirety.

A variety of mixtures of abrasives can also be used. All of the above patents regarding dental abrasives are incorporated herein by reference. The total amount of abrasive in dentifrice compositions in various embodiments may generally range from about 6% to about 70% by weight; commonly, toothpastes contain from about 10% to about 50% of abrasives, by weight of the composition. Solution, mouth spray, mouthwash and non-abrasive gel compositions of the subject invention typically contain no abrasive, although abrasive materials may be added to such compositions.

Suitable for use in various embodiments include sudsing agents that are reasonably stable and form foam throughout a wide pH range. Sudsing agents include, but are not limited to, nonionic, anionic, amphoteric, cationic, zwitterionic, synthetic detergents, and mixtures thereof. Many suitable nonionic and amphoteric surfactants are disclosed in U.S. Pat. No. 3,988,433 issued to Benedict on Oct. 26, 1976 and U.S. Pat. No. 4,051,234, issued to Gieske et al. on Sep. 27, 1977. Many suitable nonionic surfactants are disclosed by Agricola et al., U.S. Pat. No. 3,959,458 to Agicola et al., issued on May 25, 1976, both of which are incorporated herein by reference in their entirety.

Various nonionic and amphoteric surfactants may be used in various embodiments. As used herein, nonionic surfactants that may be used in various embodiments can be broadly defined as compounds produced by the condensation of alkylene oxide groups (hydrophilic in nature) with an organic hydrophobic compound which may be aliphatic or alkyl-aromatic in nature. Examples of suitable nonionic surfactants include, but are not limited to, poloxamers (sold under trade name Pluronic), polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials.

As used herein, various amphoteric surfactants that can be used in various embodiments can be broadly described as derivatives of aliphatic secondary and tertiary amines in which the aliphatic radical can be a straight chain or branched, and wherein, one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be used in various embodiments.

Various embodiments may typically comprise a nonionic, amphoteric, or combination of nonionic and amphoteric surfactant each at a level of from about 0.025% to about 5%, in another embodiment from about 0.05% to about 4%, and in even another embodiment from about 0.1% to about 3% by weight, although other ranges of such materials may be present in various embodiments.

As used herein, anionic surfactants that can be added to various embodiments include water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Various mixtures of anionic surfactants can also be employed. Some embodiments typically comprise an anionic surfactant at a level of from about 0.025% to about 9%, and in another embodiment from about 0.05% to about 7%, and in still another embodiment from about 0.1% to about 5% by weight.

Toothpastes and gels typically include a thickening agent added to the compound to create a desirable consistency, to provide desirable release characteristics when used, to increase shelf stability, and to increase the overall stability of the composition, etc. Preferred thickening agents that may be used in various embodiments include, but are not limited to, carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica may be added to further improve the texture of the composition.

Thickening agents may include, with the exception of polymeric polyether compounds, e.g., polyethylene or polypropylene oxide (M.W. 300 to 1,000,000), capped with alkyl or acyl groups containing 1 to about 18 carbon atoms.

A preferred class of thickening or gelling agents for use in various embodiments includes a class of homopolymers of acrylic acid cross linked with an alkyl ether of pentaerythritol or an alkyl ether of sucrose, or carbomers. Carbomers are commercially available from B. F. Goodrich as the Carbopol® series. Additional carbopols that may be included in various embodiments includes Carbopol 934, 940, 941, 956, and mixtures thereof.

Subgingival gel carrier for use in or around periodontal pockets may include copolymers of lactide and glycolide monomers. A typical copolymer for use in these compositions has a molecular weight in the range of from about 1,000 to about 120,000, these values are average numbers for the molecular weights of the various components. For a more thorough discussion and listing of such polymers the reader is directed to see: U.S. Pat. No. 5,198,220, issued to Damani, on Mar. 30, 1993; U.S. Pat. No. 5,242,910, issued to Damani, on Sep. 7, 1993; and U.S. Pat. No. 4,443,430, issued to Mattei, on Apr. 17, 1984, all of which are incorporated herein by reference in their entirety.

Thickening agents in an amount from about 0.1% to about 15%, or from about 0.2% to about 6%, in another embodiment from about 0.4% to about 5%, by weight of the total toothpaste or gel composition, can be used. Higher concentrations can be used for sachets, non-abrasive gels and subgingival gels.

Various embodiments may include a humectant, an additive that helps to keep various compositions such as tooth paste from hardening upon exposure to air. Additional benefits from the addition of hemectants include improved mouth feel including an enhanced moist feel to the mouth. Some hemectants may also impart a desirable sweet flavor to various compositions. A typical humectant, on a pure humectant basis, generally comprises from about 0% to about 70%, preferably from about 5% to about 25%, by weight of the compositions herein. Suitable humectants for use in various embodiments include, but are not limited to, edible polyhydric alcohols such as glycerin, sorbitol, xylitol, butylene glycol, polyethylene glycol, and propylene glycol, especially sorbitol and glycerin.

Various embodiments may also include flavoring agents. Suitable flavoring agents for use in various embodiments may include, for example, oil of wintergreen, oil of peppermint, oil of spearmint, clove bud oil, menthol, anethole, methyl salicylate, eucalyptol, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels from about 0.001% to about 5%, by weight of the composition.

Sweetening agents which can be added to various embodiments include, but are not limited to, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A typical composition may include from about 0.1% to about 10% of these agents, in another embodiment from about 0.1% to about 1%, by weight of the composition.

Various embodiments may include coolants, salivating agents, warming agents, numbing agents and analgesics. Typically, agents are included in the compositions at a level of from about 0.001% to about 10%, in another embodiment from about 0.1% to about 1%, by weight of the composition.

Coolants can be any of a wide variety of materials including materials such as carboxamides, menthol, ketals, diols, and mixtures thereof. Various coolants especially useful in the present compositions are paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional useful coolants may be selected from the group consisting of menthol, 3-1-menthoxypropane-1,2-di-ol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosures of both are herein incorporated by reference in their entirety.

Salivating agents that may be added to various embodiments include Jambu® manufactured by Takasago. Typical warming agents that may be added include, for example, capsicum and nicotinate esters, such as benzyl nicotinate. Preferred numbing agents include benzocaine, lidocaine, clove bud oil, and ethanol.

Various embodiments may include an anticalculus agent, for example, a pyrophosphate ion source from a pyrophosphate salt. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (KyP₂O₇) in their unhydrated as well as hydrated forms are the preferred species. In various embodiments at least one pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may range from about 1% to about 15%, in another embodiment from about 1.5% to about 10%, and in still another embodiment from about 2% to about 6%. Free pyrophosphate ions may be present in a variety of protonated states depending on the pH of the composition.

Compositions comprising predominately undissolved pyrophosphate commonly refer to compositions that include no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt in its solid particle form, may be in its crystalline and/or amorphous state, with the particle size of the salt preferably being small enough to be aesthetically acceptable and readily soluble during use. The amount of pyrophosphate salt useful in making these compositions is any amount effective to help control tartar; these amounts generally range from about 1.5% to about 15%, in another embodiment from about 2% to about 10%, and in still another embodiment the amount ranges from about 3% to about 8%, by weight of the dentifrice composition. Various embodiments may also include a mixture of dissolved and undissolved pyrophosphate salts. Any of the aforementioned pyrophosphate salts may be used.

Various pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated therein into Kirk & Othmer.

Optional agents to be used in place of, or in combination with, the pyrophosphate salt include materials such as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Various embodiments may also include alkali metal bicarbonate salts. Typically, alkali metal bicarbonate salts may be soluble in water and unless stabilized, they tend to release carbon dioxide in an aqueous system. Sodium bicarbonate, also known as baking soda, is an alkali metal bicarbonate salt commonly used in compositions intended for use in oral hygiene and medicines. Various embodiments may included at least one alkali metal bicarbonate salt in a range from about 0.5% to about 30%, or in a range of from about 0.5% to about 15%, and in some cases in a range from about 0.5% to about 5% of the weight of the composition.

Water employed in the preparation of commercially suitable oral compositions should preferably be of low ion content and free of organic impurities. Water generally comprises from about 5% to about 70%, and in another embodiment from about 20% to about 50%, by weight of the composition herein. These amounts of water include the free water which is added plus that which is introduced with other materials, such as with sorbitol.

Titanium dioxide may also be added to the present composition. Titanium dioxide is a white powder which adds opacity to the compositions. Titanium dioxide generally comprises from about 0.25% to about 5% by weight of the dentifrice compositions.

Antimicrobial antiplaque agents may also by optionally present in oral compositions. Such agents may include, but are not limited to, triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minocycline, and metronidazole; and analogs and salts of the above antimicrobial antiplaque agents. If present, the antimicrobial antiplaque agents generally comprise from about 0.1% to about 5% by weight of the compositions of the present invention.

Anti-inflammatory agents may also be present in the oral compositions of the present invention. Such agents may include, but are not limited to, non-steroidal anti-inflammatory agents such as aspirin, ketorolac, flurbiprofen, ibuprofen, naproxen, indomethacin, aspirin, ketoprofen, piroxicam and meclofenamic acid, and mixtures thereof. If present, the anti-inflammatory agents generally comprise from about 0.001% to about 5% by weight of the compositions of the present invention. Ketorolac is described in U.S. Pat. No. 5,626,838, issued May 6, 1997, incorporated herein by reference in its entirety.

Other optional agents include synthetic anionic polymeric polycarboxylates being employed in the form of their free acids or partially or fully neutralized water soluble alkali metal (e.g. potassium and preferably sodium) or ammonium salts and are disclosed in U.S. Pat. No. 4,152,420 to Gaffar, U.S. Pat. No. 3,956,480 to Dichter et al., U.S. Pat. No. 4,138,477 to Gaffar, U.S. Pat. No. 4,183,914 to Gaffar et al., and U.S. Pat. No. 4,906,456 to Gaffar et al., all of which are incorporated herein by reference in their entirety. Typical ratios are about 1:4 to 4:1 copolymers of maleic anhydride or acid with another polymerizable ethylenically unsaturated monomer, including methyl vinyl ether (methoxyethylene) having a molecular weight (M.W.) of about 30,000 to about 1,000,000. These copolymers are available for example as Gantrez (AN 139 (M.W. 500,000), A.N. 119 (M.W. 250,000) and preferably S-97 Pharmaceutical Grade (M.W. 70,000), of GAF Corporation.

Some embodiments selectively include H-2 antagonists including compounds disclosed in U.S. Pat. No. 5,294,433, Singer et al., issued Mar. 15, 1994, which is herein incorporated by reference in its entirety.

Various embodiments may also relate to methods of recrystallizing and/or remineralizing enamel and/or dentine in humans or lower animals in need thereof, by administering an effective amount of the compositions of various embodiments described herein, to the oral cavity by application methods described, for example, throughout and below.

A safe and effective amount of the compositions of various embodiments may be topically applied to the mucosal tissue of the oral cavity, to the gingival tissue of the oral cavity, and/or to the surface of the teeth, for the treatment or prevention of the above mentioned conditions of the oral cavity, in any of several conventional ways some of which are described as follows. For example, the gingival or mucosal tissue may be rinsed with a solution (e.g., mouth rinse, mouth spray); or in a dentifrice (e.g., toothpaste, tooth gel or tooth powder), the gingival/mucosal tissue and/or teeth are bathed in the liquid and/or lather generated by brushing the teeth. Other non-limiting examples include applying a non-abrasive gel or paste, directly to the gingival/mucosal tissue or to the teeth with or without an oral care appliance described below. Preferred methods of using the compositions of this invention are via rinsing with a mouth rinse solution and via brushing with a dentifrice.

For the method of treating diseases or conditions of the oral cavity, including caries, a safe and effective amount of the present compositions are preferably applied to the gingival/mucosal tissue and/or the teeth (for example, by rinsing with a mouth rinse, directly applying a non-abrasive gel with or without a device, applying a dentifrice or a tooth gel with a toothbrush, etc.) preferably for at least about 10 seconds, in another embodiment from about 20 seconds to about 10 minutes, in even another embodiment from about 30 seconds to about 60 seconds. The method often involves expectoration of most of the composition following such contact. The frequency of such contact is preferably from about once per week to about four times per day, in another embodiment from about thrice per week to about three times per day, in even another embodiment from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. If delivery to the periodontal pockets is desirable, a mouth rinse can be delivered to the periodontal pocket using a syringe or water injection device. These devices are known to one skilled in the art. Devices of this type include “Water Pik” marketed by Teledyne Corporation. After irrigating, the subject can swish the rinse in the mouth to also cover the dorsal tongue and other gingival and mucosal surfaces. In addition, toothpaste, non-abrasive gel, tooth gel, etc., can be brushed onto the tongue surface and other gingival and mucosal tissues of the oral cavity. The period of such treatment typically ranges from about one day to a lifetime. The subject may repeat the application as needed. The duration of treatment is preferably from about 3 weeks to about 3 months, but may be shorter or longer depending on the severity of the condition being treated, the particular delivery form utilized and the patient's response to treatment.

The compositions of this invention are useful for both human and other lower animal (e.g. pets, zoo, or domestic animals) applications. Accordingly, the following examples and discussion are presented by way of guidance and explanation and not limitation.

### EXAMPLES

In some studies lesioned bovine enamel specimens were used to test the efficacy of some of these compositions. The close similarities in mineral distribution and structure of enamel support the use of bovine specimens in remineralization studies of artificial caries virtually ensures that what is learned in these studies can be readily applied to humans. Lesioned bovine enamel was used because it is an excellent model for human enamel and its use is less expensive and entails fewer ethical considerations than the use of similar materials derived from humans.

Evidence of their efficiency is also presented. Experimental evidence in support of these assertions includes experiments including the following steps: (1) an alloy is formed by alloying tricalcium phosphates and at least one metal oxide, (2) Alloyed Tricalcium Phosphate (ACP) compositions according to various embodiments were contacted with boney or enameled surfaces or, for example, lesioned bovine enamel in order to study the remineralization of the surfaces contacted with the alloy; and (3) the effectiveness of ACP materials were assessed by measuring the microhardness of tooth or bone surfaces contacted with various forms of ACP.

### Example 1

### Preparation and Test of Active Ingredients

Amorphous tricalcium phosphate (Ca₃(PO₄)₂) was prepared as follows. Equal amounts of calcium carbonate (CaCO₃) and calcium phosphate dihydrate (CaHPO₄.2H₂O) were loaded into a crucible and heated to 1050° C. for 24 hours in a muffle furnace. Following a room temperature quench the resultant Ca—P product was determined by x-ray diffraction to be crystalline beta-tricalcium phosphate (beta-TCP, JCPDS # 09-0169). The product was then loaded into a 150 ml stainless steel jar along with 25, 10 mm stainless steel balls, 2 mL of ethanol was added to reduce caking. The jar and contents were then weighed and placed into a PM100 planetary ball mill. The powder was pulverized unidirectionally at 450 Rpms for 25 hours. After the milling period the powder was evacuated at 10-3 Torr in a vacuum oven at room temperature to evolve the added ethanol. The powders were determined to be largely amorphous and microcrystalline via x-ray diffraction. ACP-TiO₂ nanopowders (denoted, for example, as ACP95 or ACP95 (% wt. % TiO₂) were alloyed by the same milling procedure.

Briefly, a mixture of for tricalcium phosphate and a metal oxide such as TiO₂ was placed in a 150 ml stainless steel vessel. In order to make an alloy comprising, for example, 90 wt. % tricalcium phosphate and 10 wt. % TiO₂ the ratio of tricalcium phosphate to metal oxide in the mixture added to the vessel was on the order of about 10 to 1. The vessel also included 25, 10 mm balls and about 2-5 ml of ethanol added to prevent caking. The vessel was sealed and placed in a PM 100 ball mill and the mixture was pulverized, unidirectionally at about 450 rpm for 25 hours.

Similar methods can and were used to make alloys according to various embodiments. One a threshold of energy input and contact time is reached to effect the solid state transformation of distinct compounds, such as tricalcium phosphate and metal oxides into an alloy of the compounds, it is possible to adjust milling conditions to create alloys in having different particle sizes and distributions of particle size.

Bovine enamel specimens (3 mm) were ground and polished to a high surface luster with Gamma Alumina using standard methods. Six specimens per group were prepared for this study. Artificial white-spot lesions were formed in the enamel specimens by a 30-hour immersion into a solution of 0.1 M lactic acid and 0.2% Carbopol C907 which had been saturated with hydroxyapatite and adjusted to pH 5.0 at 37° C. The lesion surface Vickers hardness ranged between 20 and 45.

Artificial lesions were formed in the enamel specimens by a 30-hour immersion in a solution of 0.1 M lactic acid and 0.2% CArbopol C907 which was saturated with hydroxyapatite and adjusted to pH 5.0 at 37° C. Initial surface hardness was measured, it typically ranged from between about 20 to about 45 (units); the depth of the average lesion was about 72 microns.

Specimens were treated as follows. For each group a stopper loaded with six specimens was immersed in a solution comprising 10 ml artificial saliva and the appropriate amount of dentifrice (solute+5 ml polyethylene glycol (PEG)). Fresh solutions were stirred for approximately one minute prior to use. There were a total of four treatments per day for five days, with the exception of the pellicle forming treatment on the first day. The treatments were magnetically agitated, lasting one minute and then immersed in either agitated artificial saliva or subjected to an acid challenge when not exposed to a remineralization solution. Saliva was changed once daily after the demineralization step.

Specimens were subjected to six different conditions. These conditions are follows: 1) negative control, water; 2) positive control, dentifrice unstabilized solution including 0.0882 g CaCl₂.H₂O and 0.054 g KH₂PO₄; 3) ACP100, (No TiO₂) unstabilized solution including 0.062 g of ACP; 4) stabilized ACP preparation including 0.0597 g of ACP95, (ACP alloyed with 5 wt. % TiO₂); 4) stabilized ACP preparation including 0.0594 g of ACP90, (ACP alloyed with 10 wt. % TiO₂); and 4) stabilized ACP preparation including 0.0551 g of ACP85, (ACP alloyed with 15 wt. % TiO₂). With the exception of group 1 (water only) each group includes 30 mM Ca⁺² and 20 mM PO₄⁻³.

All samples were subjected to a cyclic treatment procedure that included exposing the samples to conditions known to attack enamel and mineralizing preparation as described in groups 1-6 in the above. Typically, a cyclic treatment procedure consists of a 4.0 hour/day acid challenge in the lesion forming solution and four, one-minute remineralization solution treatment periods. A pellicle is developed prior to solution treatment by exposing the specimens to saliva for about one hour. After the treatments, the specimens were rinsed with deionized water and placed back into the saliva. For the remaining time the specimens were kept in artificial saliva.

The regimen was repeated for a total of 5 days. The treatment schedule used for this experiment is as follows: (a) 8:00-8:01 a.m., solution treatment*; (b) 8:01-9:00 a.m., saliva treatment; (c) 9:00-9:01 a.m., solution treatment; (d) 9:01-10:00 a.m., saliva treatment; (e) 10:00 a.m.-2:00 p.m., acid challenge; (f) 2:00-3:00 p.m., saliva treatment; (g) 3:00-3:01 p.m., solution treatment; (h) 3:01-4:00 p.m., saliva treatment; (i) 4:00-4:01 p.m., solution treatment; (j) 4:01 p.m.-8:00 a.m.(next day), saliva treatment; and (k) Back to (a). *On the first day, this treatment was not given; the test was initiated by exposing the enamel specimens to saliva for one hour to permit pellicle development prior to any treatments. ‡ Denotes points in the study when the fresh saliva was changed.

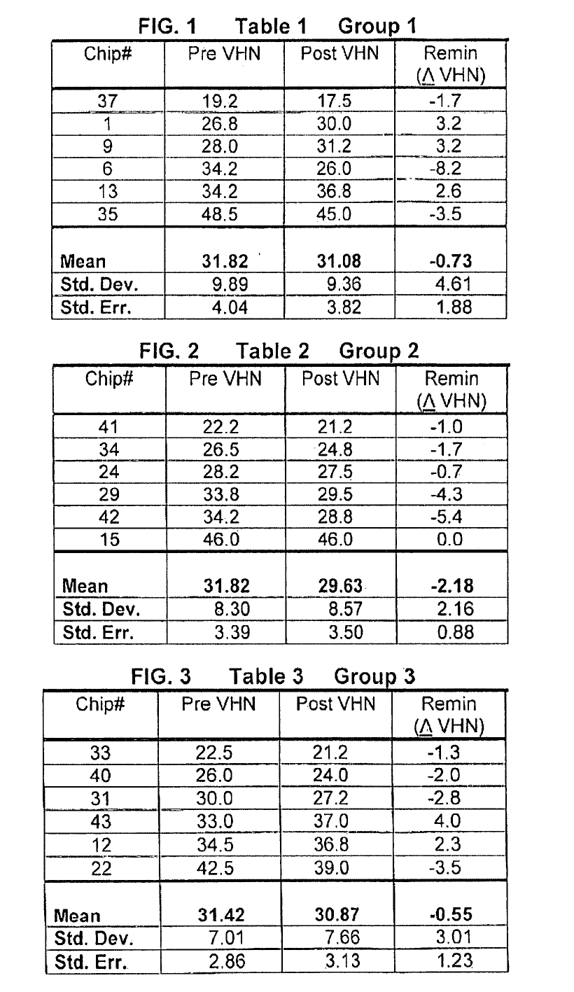

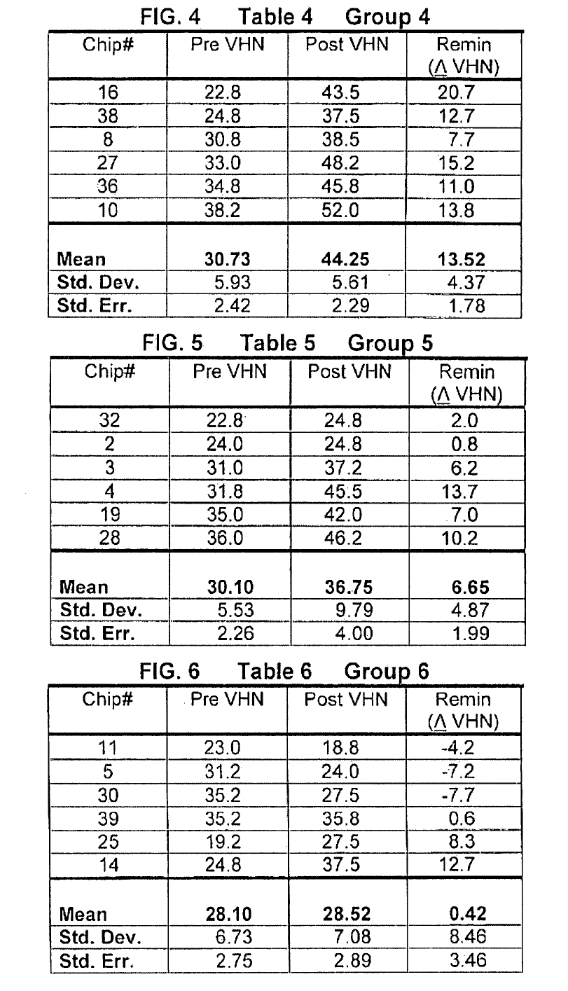

The degree of remineralization was determined by comparing Vickers surface microhardness (VHN) unless before and after treatment of the enamel specimens. The mean and standard deviations were calculated and analyzed for significant differences for each of the six samples subjected to the six different remineralizing conditions (groups 1-6). These values are reported in Tables 1-6 (FIGS. 1-6) for groups 1-6 described above. The experimental results and discussion are as follows.

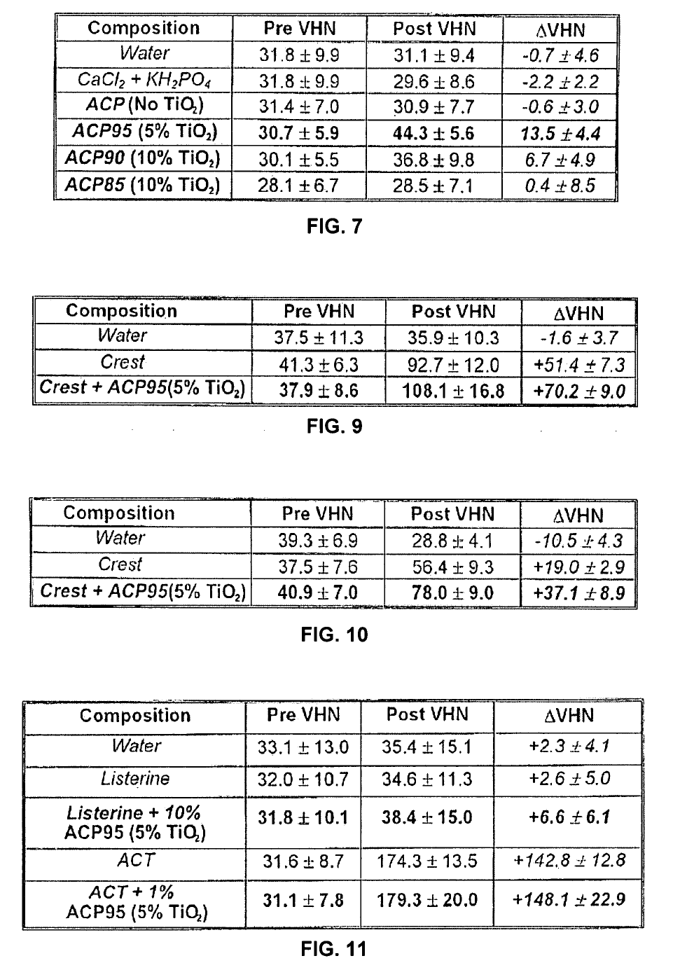

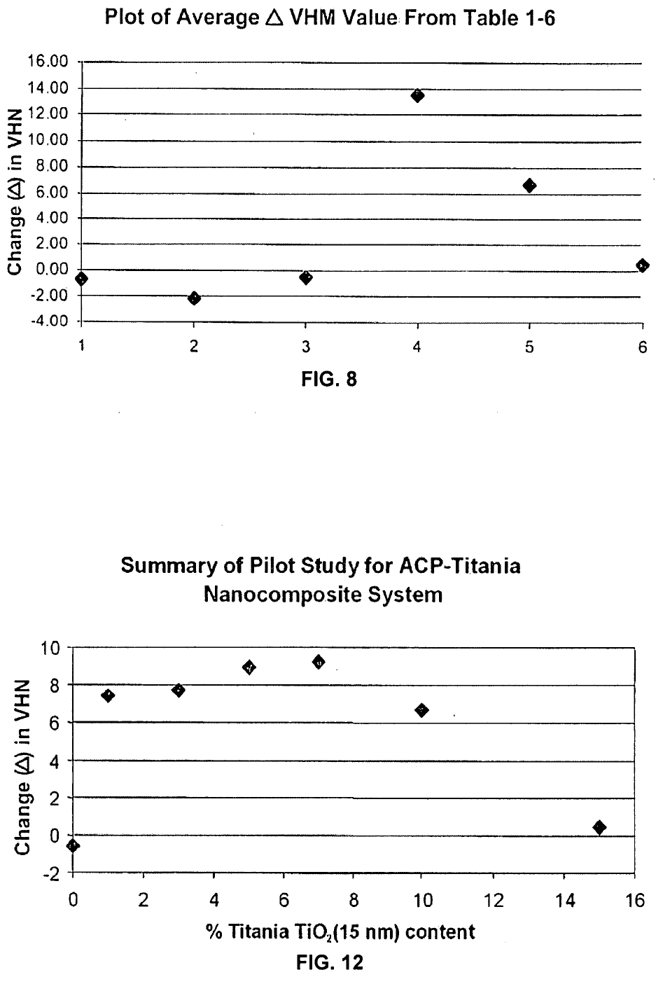

The pre, post and change in Vickers microhardness are presented in FIGS. 1-7 including initial data collected in various remunerating preparations (FIGS. 1-6). The mean values for change in VHN values from each group are summarized in Table 7 (in FIG. 7). The mean values for change in VHN values for each group are illustrated graphically in FIG. 8.

These data indicate that including remineralizing preparations which included alloys such as ACP95 (group 4), ACP90 (group 5), and ACP85 (group 6), were more effective than preparations of water alone (group 1), CaCl₂.H₂O and KH₂PO₄ (group 2) or unalloyed amorphous tricalcium phosphate (ACP-No TiO₂) ACP (group 3) in effectively remineralizing enamel. The mechanical alloying process appears to enhance the interface between the amorphous tricalcium phosphate and the titania nanopowder thereby forming an alloy of these compounds. The most effective composition appears to be ACP95, which comprises 5% TiO₂. The fall-off at higher titania content could be attributed to a reduction in nanoscale properties due to the formation of larger clusters of nanoparticles which may tend to occur at higher concentrations of the material. At the other extreme, when too little titania is present in the composition, there is not enough interfacing between titania and amorphous tricalcium phosphate to fully exploit the properties of the material. The unalloyed compositions in Groups 2 and 3 do not promote effective remineralization, presumably due to the formation of calcium-phosphate complexes.

### Example 2

Testing of the effect of exposing enameled surfaces to various compositions including some comprising ACP alloyed with TiO₂ to a dentifrice which already included fluoride.

ACP alloys of tricalcium phosphate and TiO₂ were prepared as outlined in Example 1. Separate studies were performed using ACP and various controls in artificial saliva and pooled human saliva. The results are as follows:

Crest® toothpaste, a registered trademark of Proctor and Gamble, was used, as commercially available, as a control and modified to include various experimental remineralizing compositions. The various treatment groups tested were as follows: group 1): negative control: water; group 2): positive control: Crests); group 3): binary alloy: ACP, 1% TiO₂; group 4): binary alloy: ACP, 3% TiO₂; group 5): binary alloy: ACP, 5% TiO₂ (fresh batch); and group 6): binary alloy: ACP, 7% TiO₂. With the exception of water, each group included about 30 mM Ca⁺² and about 20 mM PO₄⁻³. Two sets of tests were carried out. In one test the samples were exposed to artificial saliva, in a similar, test the samples were exposed to pooled human saliva.

The pre, post and change in Vickers microhardness results of the pilot study are presented (see FIGS. 9 and 10). Table 8 (FIG. 9) summarizes results from artificial saliva studies, while Table 9 (FIG. 10) summarizes results from tests conducted using pooled human saliva. Each separate study illustrates the potential of adding ACP95 to formulations such as Crest®, which contains on the order of about 1100 ppm fluoride. The relative increase in micorhardness for Crest®D+ACP95 over Crest® in tables 8 and 9 (figure and 9 and 10) is about 27% and about 48%, respectively. These values indicate that the mechanical alloying process used to manufacture ACP enhances the degree of interfacing between tricalcium phosphate and titania nanopowder. The interfacing process inhibits the ability of calcium in ACP to bond with free fluoride, which is generally a problem that has been addressed by compartmentalized dentifrice systems (individual tubes, one for tricalcium phosphate and one for sodium fluoride), which are required in conventional preparations of fluoride and calcium, due to the high affinity of calcium and fluoride to bind to one another. Thus, combing ACP with preparations that include fluoride appears to offer a method for promoting the coexistence of fluoride and calcium in dental preparations.

Referring now to FIG. 11, the change in VHN values of this study were combined with the previous study and are plotted together. Although not shown, the error bars at each point were similar to those expressed in the data found and presented in tables 7, 8 and 9. These results, illustrate reproducibility of the mechanical alloying process. These results can be used to estimate which compositions will be most efficacious in remineralizing enamel specimens based on their ability to increase the VHN values.

### Example 3

Mouth rinses, including some supplemented with various types of ACP, were tested to determine their effect on the integrity of enamel in the face of acid challenge. The same test sample preparations, methods of preparing the ACP-TiO₂ nanopowders and enamel specimens that were used in the toothpaste protocol were used in the mouth rinse studies. Commercially available mouth rinses, Listerine®, a registered trademark of Pfizer, and ACT®, a registered trademark of Johnson & Johnson, were used as positive controls. They were also supplemented with various levels of an alloy of tricalcium phosphate and TiO₂. The mouth rinse studies were carried out using mixtures of artificial saliva and pooled human saliva; specifics of the protocol and results were as follows:

The specimens were treated as follows. For each group a stopper loaded with six specimens was immersed in a solution comprising 5 ml artificial saliva and 10 ml mouth rinse. The manufacturers of ACT®, a fluoride-containing rinse (0.05% NaF), recommend exposing the oral cavity to 10 ml of the mouth rinse for 1 minute, while the manufacturer of Listerine® recommends using 20 ml for 30 seconds. To simplify the study, 10 ml of Listerine® were used over a 1 minute test period. Solutions with ACP included the following: (0.1 grams ACP95), 5 (0.5 grams ACP95) or 10 (10 grams ACP95) wt. % ACP95. Fresh solutions were stirred for approximately one minute prior to use. There were a total of four treatments per day for five days. Specimens were immersed in pooled human saliva the night prior to the first day of the experiment in order to form a pellicle. The treatments were magnetically agitated, lasting one minute and then immersed in either agitated or pooled human saliva prior to an acid. Saliva was changed once daily after the demineralization step.

The groups were arranged as follows; group 1, negative control, water; group 2, Negative Control, Listerine®; group 3, test, Listerine®+10% ACP95; group 4, positive control, ACT®; and group 5, test, ACT+1% ACP95.

The cyclic treatment procedure consisted of a 4.0 hour/day acid challenge in the lesion forming solution and four one-minute remineralization solution treatment periods. After treatments and challenges, specimens are immersed in pooled human saliva. Industry accepted artificial saliva was used in this example. The regimen was repeated over the course of 5 days. The treatment schedule used for this experiment was as follows: (a) 8:00-8:01 a.m., rinse; (b) 8:01-9:00 a.m., saliva; (c) 9:00-9:01 a.m., rinse; (d) 9:01-10:00 a.m., saliva; (e) 10:00 a.m.-2:00 p.m., acid challenge; (f) 2:00-3:00 p.m., saliva‡; (g) 3:00-3:01 p.m., rinse; (h) 3:01-4:00 p.m., saliva; (i) 4:00-4:01 p.m., rinse; and (j) 4:01 p.m.-8:00 a.m.(next day), saliva. ‡ Denotes when fresh saliva changed.

### Example 4

### Results of Treating Enamel Surfaces with Mouth Rinses that Include ACP-SiO₂

The pre, post and change in Vickers microhardness are presented in Table 10 (FIG. 12). The incorporation of ACP95 into Listerine® produced a level of remineralization approximately three times higher than did the base Listerine® formulation, despite using a sub optimal level of Listerine®. For ACT®, the best remineralization results were obtained when 1% of ACP95 was added to the mouth rinse. These studies show that a higher content of ACP95 drives the formation of calcium fluoride. This is likely due to the reaction between elevated amounts of calcium and available fluoride in ACT®. This interaction inhibits remineralization potentially by lowering the level of the formation of bioavailable calcium and fluoride available for mineralizing enamel. Accordingly, preparations including less than 1% ACP are likely to promote enhanced remineralization over base ACT® formulations.

### Example 5

### Testing Efficacy of Including Alloys of Tricalcium Phosphate and Metal Oxides in Chewing Gums for Preventing Caries.

Commercially available chewing gums both “as sold” and supplemented with remineralizing compositions were tested to determine the effect of exposing bovine enamel samples to preparations including these gums. Trident®, a registered trademark of Cadbury Schwepps, and Wrigley's® chewing gum, a registered trademark of Wrigley's, were purchased and used as detailed below.

ACP and enamel specimens used in this example were prepared by the same methods described in the previous example.

Sugar-free mint flavored Trident® and Trident® Whitening w/Recaldent were purchased from a local store. The Trident® gum was pink and soft and a single piece weighted about 1.76 grams. The Trident® Whitening weighted 1.44 grams and was white with a hard ‘casing’. The gum was grated using a conventional kitchen grater in order to obtain small particles and increase the surface area of the gum. Sugar-free mint Trident® was divided into two masses, one for use as a control and the other to be combined with ACP95 (5 wt. % TiO₂).

Doublemint Wrigley's® sticks were purchased from a local store. The gum was dull gray and each piece weighed approximately 3.18 grams. The gum was grated using a conventional kitchen grater in order to obtain small particles and increase the surface area of the gum. Doublemint Wrigley® gum was divided into two masses, one for use as a control and the other to be combined with ACP95 (5 wt. % TiO₂).

Specimens of bovine enamel were treated as follows. For each group of specimens a stopper loaded with six specimens was immersed in mixtures comprising 15 ml artificial saliva and 1.44 grams of each gum. Mixtures supplemented with ACP TiO₂ included 10 mg of ACP95 with a calcium level of 667 ppm (in 15 ml volume). Fresh mixtures were stirred for approximately one minute prior to use in the study. A total of four treatments per day for four days were carried. Specimens were immersed in pooled human saliva the night prior to the initial experiment day in order to form pellicle. Due to the gummy nature of the preparations, the treatment cups with specimens were placed in larger cups and situated in a rotating platform for agitation. Samples were immersed in agitated pooled human saliva prior to acid challenge. After the challenge specimens were again immersed in pooled human saliva, followed by treatment with a preparation that included one form of the gum. Saliva was changed once at the end of the 2^{nd} acid challenge (prior to third treatment with a gum preparation).

The groups tested in this study were as follows: group 1, negative control, water; group 2, positive control, sugar-free mint Trident®; group 3, test, sugar-free mint Trident®+ACP95; group 4 positive control, Trident® Whitening w/Recaldent; group 5, positive control, Wrigley's Doublemint®; and group 6, test, Wrigley's Doublemint®+ACP95.

The samples were subjected to a cyclic treatment procedure consisting of three 20 minute white-spot challenges (without Carbopol for diminished attack) and four, one hour long gum treatment periods. The initial pellicle was developed the night prior to the first day of the initial experiment. After treatments and challenges specimens were immersed in pools of human derived saliva. The cycle was repeated for a total of 4 days. The following treatment schedule was used for this experiment: (a) 8:00-9:00 a.m., gum; (b) 9:00-9:30 a.m., saliva; (c) 9:30-9:50 a.m., acid challenge; (d) 9:50-10:20 a.m., saliva; (d) 10:20-11:20 a.m., gum; (e) 11:20-11:50 a.m., saliva; (f) 11:50 a.m.-12:10 p.m., acid challenge; (g) 12:10-12:40 p.m., saliva ‡; (h) 12:40-1:40 p.m., gum; (i) 1:40-2:10 p.m., saliva; (j) 2:10-2:30 p.m., acid challenge; (k) 2:30-3:00 p.m., saliva; (l) 3:00-4:00 p.m., gum; and (m) 4:00 p.m.—overnight, saliva. ‡ Denotes when fresh pooled human saliva changed.

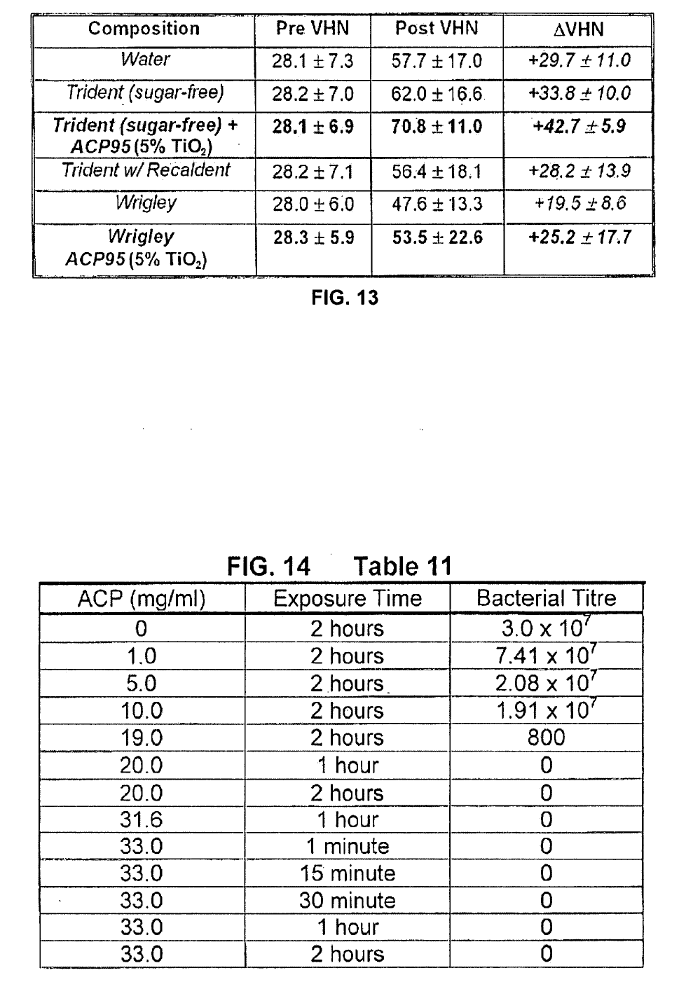

The Vickers hardness of the samples was measured pre and post treatment. The results of these studies study are summarized in table 11 (FIG. 13). These results indicate that muted acid challenges resulted in overall remineralization even in the negative controls. However, the incorporation of ACP95 into Trident® produced a substantial remineralization of about 21% relative to the effect of gum formulation comprising Trident® alone.

Referring again to FIG. 13, the improvement over Trident® supplemented with Recaldent was even larger. Gum supplemented with Recaldent is thought to remineralize enamel through stabilization of calcium-phosphate through CPP, a milk-based protein. However, the Trident® formulation combined with ACP95 provided a mean improvement of approximately 34% over Trident® combined with Recaldent. This is consistent with ACP enhancing remineralizing in excess of the results obtained with the milk protein derivative Recaldent.

Significant remineralization was not observed when a sugar based gum such as Wrigley's® was used. One possibility is that sugar in the gum may feed active cariogenic bacteria remaining in the pooled human saliva and these bacteria may attack the enamel surface. The presence of ACP95 in the composition enhances the tendency towards remineralization as shown in table 5 (FIG. 5). Taken together these experimental results demonstrate that adding ACP95 to gum improves gum-based remineralizing.

### Example 6

### Testing Efficacy of Amorphous Tricalcium Phosphate as an Antimicrobial Agent.

Amorphous Tricalcium Phosphate, milled, was produced through a solid-state ball milling procedure, and its antimicrobial activity was measured as follows:

The antimicrobial activity of ACP was tested on five strains of oral bacteria which have been shown to contribute to the development of caries. These strains include: Streptococcus mutans TH16, Lactococcus casei, Actinomyces naeslundii, Streptococcus sanguis, and Streptococcus salivarius. Cultures of each of these strains were grown in tryptic soy broth (TSB) at 37° C., 5% CO₂ for 20 hours. Each culture was then pelleted by centrifugation for 20 minutes at 3,000 g, after which the supernatant was removed and discarded. The remaining pellet was then re-suspended with 20 ml fresh TSB. A master culture was formed by combining all five solutions of 20 ml each (100 ml total). From this master solution, 2 ml was extracted and placed in fresh tubes containing 28 ml fresh TSB. Amorphous tricalcium phosphate was added to this fresh culture to achieve the appropriate final concentration. The form of amorphous tricalcium phosphate used in this example was manufactured using the same methods described earlier except that the milling process was performed for 5 days instead of the pervious 25 hour time period. In order to minimize experimental error in the data, the experiment was repeated multiple times on separate days.

The effect of both the concentration and length of exposure time of the amorphous tricalcium phosphate on antimicrobial activity was evaluated. Cultures to be examined were placed on a shaker platform and incubated 37° C. for 24 to 48 hours prior to determination of the bacterial titre.

Referring now to FIG. 14 (table 7), as these data illustrate, the antimicrobial activity of amorphous tricalcium phosphate is evident in concentrations as low as 2 mg/ml of amorphous tricalcium phosphate in the bacterial culture. The material affects bacteria growth in as little time as about one minute when used at a concentration of 33 mg/ml.

Similar experiments were carried out with amorphous tricalcium phosphates milled for about 24 hours. The materials formed at the shorter milling times were not nearly as effective as an antimicrobial agent (data not shown) as the material found by milling for about 120 hours. These data indicate that the average size of the particles in the composite may have a profound impact on the biological properties of the material. And as the materials milled for 5 days, as opposed to the material milled for 24 hours, is likely of different particle size these data indicate that adjusting the particle size by, for example, changing milling times or milling methods, is likely to affect the bioactivities of the materials produced.

### Example 7

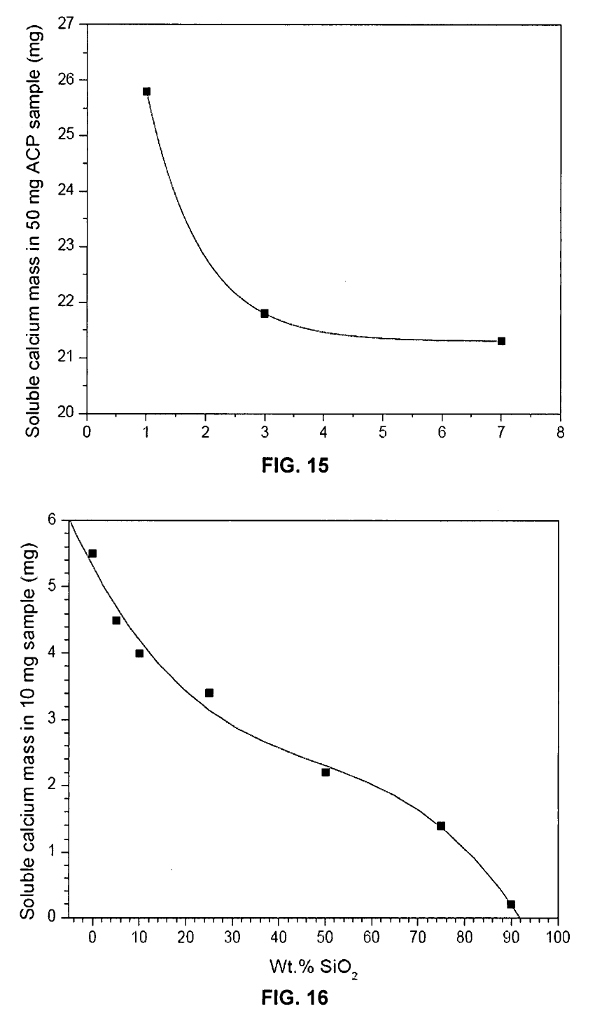

Referring now to FIG. 15, tricalcium phosphate was milled continuously for up to 7 days. Briefly the material was processed as follows: 5 ml of ethanol was added to a 150 ml stainless steel milling vessel (to prevent the powder from caking to the walls of the milling vessel and balls) 20 grams of tricalcium phosphate and 24 stainless steel balls, each ball had a diameter 10 mm were also added to the vessel. Once loaded the vessel was placed into a planetary ball milling machine and milled for up to 7 days at 350 rpms. Samples were removed from the milling vessel every 24 hours, placed in vials and set aside for analysis. The samples were analyzed for calcium solubility by dissolving (e.g. 30 mg) of the milled powder in water. The water was then analyzed for calcium content using Atomic Absorption Spectroscopy. The results of these tests are illustrated in FIG. 15.

Referring now to FIG. 16, ACPS was prepared and the material was analyzed to determine the level of water solubility of calcium in the composition as a function of the weight percent SiO₂ in the material. Briefly, ACPS was prepared by adding about 20 ml of pentane (to prevent the powder from caking to the walls of the milling vessel and balls), 24 stainless steel balls, each ball having a diameter of 10 mm, along with 20 total grams of tricalcium phosphate plus SiO₂ The experiment was repeated at various levels of silica over the range of between about 0 to about 90 wt. % SiO₂ of the total amount of solid loaded into the ball mill vessel. Once loaded, the milling vessel was placed into a planetary ball milling machine and milled for 24 hours, the mill was operated at 450 rpms.

Samples were drawn after 24 hours and assayed to determine the level of water soluble calcium in each sample. Samples of the various powders were placed in the same volume of water. The water was analyzed to determine the level of soluble calcium by the same methods outlined in reference to FIG. 15. Referring again to FIG. 16, the solubility data collected in this experiment was plotted as a function of SiO₂ content. These data fit best to a third-order polynomial signifying that there are three distinct regions, with a given composition falling into one of these regions. These results do not show a linear relationship between the amount of SiO₂ used to make the composite and the amount of soluble calcium associated with each composite. These data indicate that the blending of tricalcium phosphate with silica produces a true composite material i.e. a material that behaves differently from a simple mixture of tricalcium phosphate and SiO₂.

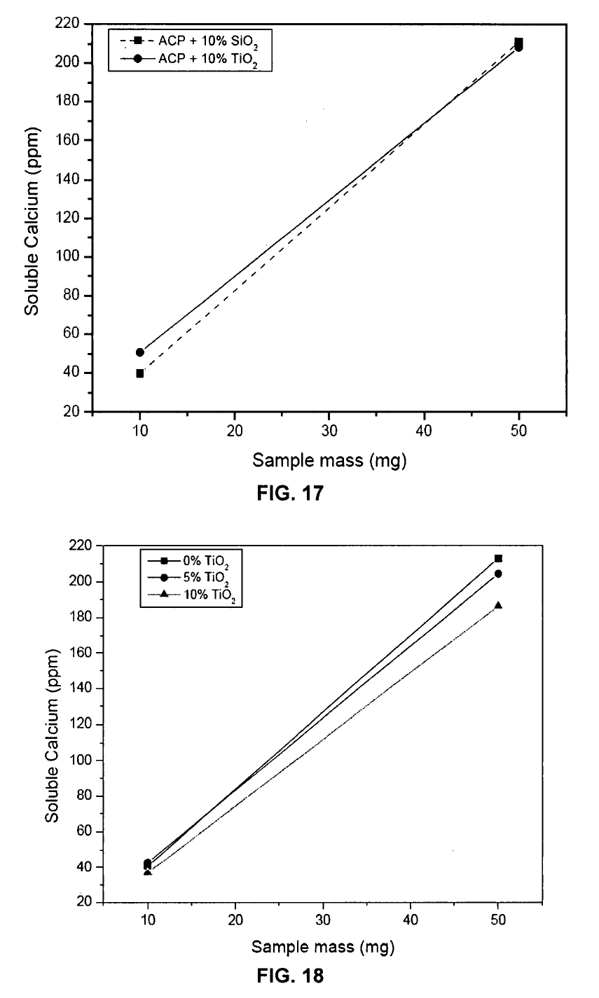

Referring now to FIG. 17, briefly, amorphous tricalcium phosphate was alloyed with either 10 wt. % TO₂ or SiO₂. The materials were prepared by adding the following materials: added to a 150 ml stainless steel milling vessel, which included 24 stainless steel balls each ball having a diameter 10 mm, 20 ml of pentane (added to prevent powder from caking to the walls of the vessel and balls), 20 grams of tricalcium phosphate plus either 10 wt. % SiO₂ or TiO₂. Once the vessel was loaded, it was placed into a planetary ball milling machine and milled for 24 hours. The mill was operated at 450 rpms under ambient conditions.

Next the solubility of calcium was measured for the two alloys. One alloy comprised amorphous tricalcium phosphate alloyed with 10 wt. % silica, and the other alloy comprised amorphous tricalcium phosphate alloyed with 10 wt. % TiO₂. Samples of each alloy were drawn and added to water. Assays were run using two different powder masses (10 and 50 mg) for each alloy added to equal volumes of water. The results were plotted in FIG. 17. Soluble calcium is plotted versus the mass of the alloy added to the analyzed water sample. These results, summarized graphically in FIG. 17, illustrate that there is no significant difference between the effect of either titania or silica on the level of water soluble calcium associated with a given alloy of amorphous tricalcium phosphate and metal oxide. Given these results, it is appears as though any metal oxide can be alloyed with amorphous tricalcium phosphate under the proper conditions to form an alloy comprising amorphous tricalcium phosphate and at least metal oxide useful for adding soluble calcium to a given system.

Referring now to FIG. 18, briefly, tricalcium phosphate was alloyed with the following levels: 0, 5, or 10 wt. % TiO₂. The alloys were prepared as follows: briefly, the following materials: were added to a 150 ml stainless steel milling vessel, 20 ml of pentane (added to prevent powder from caking to the walls of the vessel and balls), 20 grams of tricalcium phosphateplus and either 0, 5, or 10 wt. % TO₂. The vessel also included 24 stainless steel balls each ball had a diameter of 10 mm. Once the vessel was loaded, it was placed into a planetary ball milling machine and milled for 24 hours. The mill was operated at 450 rpms.

Two samples, one including 10 mg of the material and one including 50 mg of the material, were taken for each alloy and added to the same volume of water. The faction of soluble calcium was measured for each sample. Referring now to FIG. 18, the level of soluble calcium was plotted as a function of sample mass. The data were fit to a line in order to estimate the amount of soluble calcium over the range of 10 and 50 mg. of the material.

### Example 8

Surprisingly, we have shown that mechanochemical (MC) ball milling is an excellent method for creating alloys of tricalcium phosphate and metal oxides. This technique works well for several reasons, including the ease at which one can produce industry-scale quantities of these alloys at an economical price.

Additionally, we have shown that the addition of these alloys to various preparations can increase the remineralization efficacy of enamel lesions. This is also true of the remineralization process when carried out in the presence of multiple ions such as calcium, phosphate, and fluoride as compared to fluoride alone. Generally, unalloyed tricalcium phosphate cannot be efficaciously added to fluoride in a single-system composition. However, we realized that an alloy of tricalcium phosphate, with a metal oxide according to various embodiments, provides a material that does not significantly bind to or negatively interfere with fluoride.

In one embodiment, calcium phosphate-silica alloy SiO₂ (ACPS) were formed by MC ball milling tricalcium phosphate and SiO₂ (15 nm) in pentane for about 2 hours at about 550 rpms. Because the material experienced significant impact forces through ball-particle, particle-particle, and particle-wall collisions during the MC process, modifications to phosphate microstructure are likely to have occurred. We investigated this using IR spectrometry.

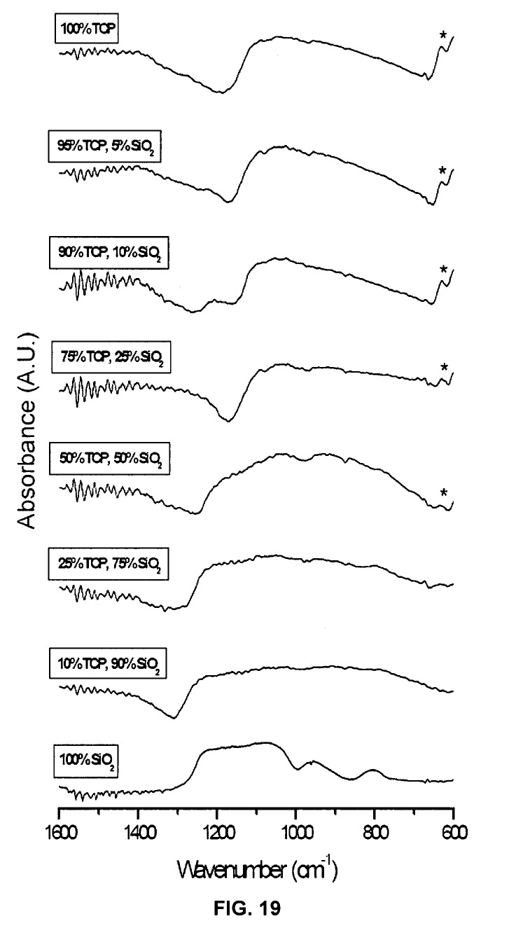

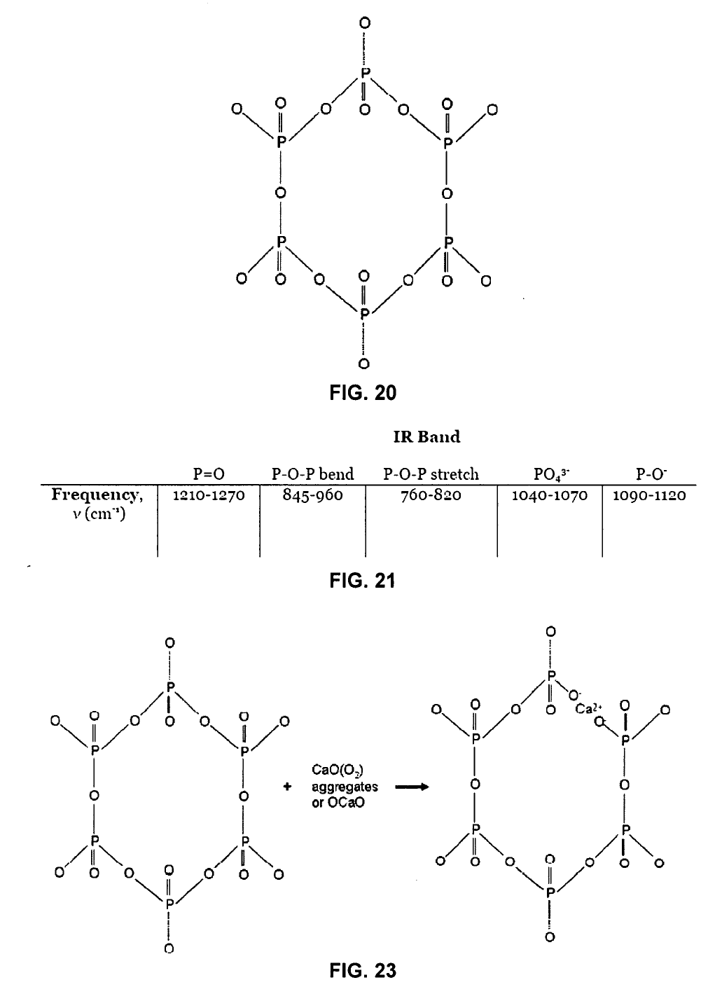

Referring now to FIG. 19, this figure shows IR spectra of various ACPS powders measured for alloys that included different levels of silica. The trend illustrated by the spectra in FIG. 19 shows: 1) modulations in P—O vibrations corresponding to an amorphous P₂O₅ network (FIG. 20) and ionic PO₄³⁻ and 2) the presence of CaO moieties in the material (as indicated by the asterisks). P-O vibrations native to covalent and ionic phosphorous structures lie principally between 700 and 1300 cm. Within this frequency range there are five principal bands, three marking covalent bonding (P═O stretch, P—O—P bend, and P—O—P stretch) and two for ionic bonding (i.e. PO₄³⁻ and P—O⁻). The overlapping of all these bands illustrated in FIG. 19 produces the broad line-shapes and is consistent with the formation of a disordered material. Interestingly, the well-defined spectral features marked with asterisks indicate these materials retain some ordered structure despite the powerful crushing forces experienced by the powder during the MC process. These features are characteristic of CaO chemical entities (either CaO(O₂) or OCaO aggregates) and will be discussed in greater detail momentarily.

While the spectra in FIG. 19 illustrate the qualitative differences in the raw data, deconvolution of the spectra using the minimum number of Lorentzian line shapes and a fifth-order baseline polynomial enabled us to make quantitative comparisons among the IR-active vibrational modes. Using published phosphate material IR data as a reference and deconvoluting the spectra in FIG. 19, the identity and center positions of the five principal characteristic bands were determined and are summarized in FIG. 21.

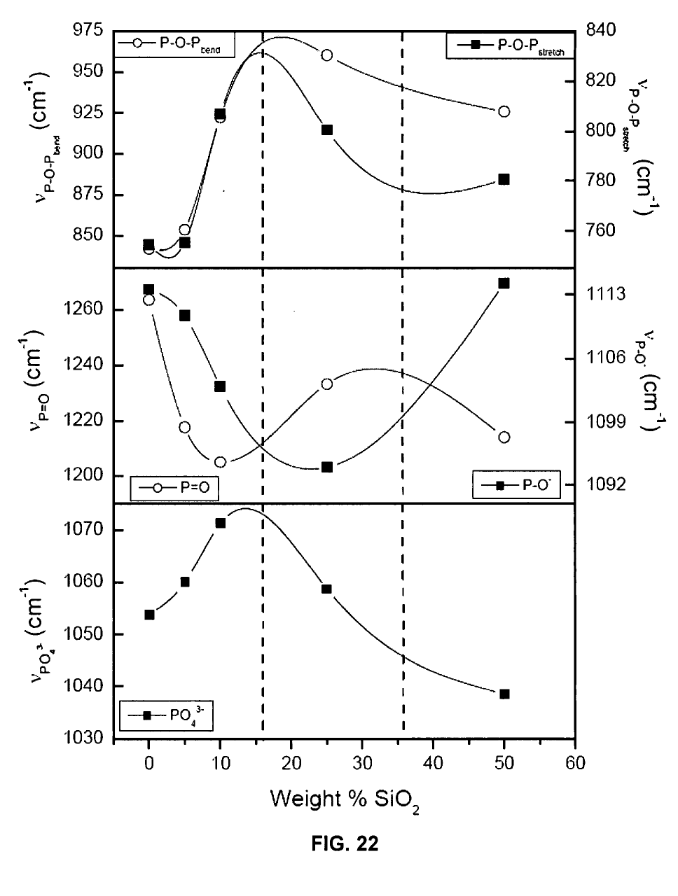

Because interpreting the width of the Lorentzian line shapes can be subjective, after peak deconvolution, assessments of the fitted line shape center of mass offers a more objective form of analysis and is therefore the technique that we chose to interpret these data. Peak centers of each of the five vibrational modes gleaned from the data in FIG. 21 are plotted against SiO₂ content. Referring now to FIG. 22, these data plotted as a function of the weight of SiO₂ in the material illustrate the microscopic effect that SiO₂ appears to have on the tricalcium phosphate phase of ACPS.

Referring now to FIG. 19, these spectra indicate significant SiO₂ character beyond 50 wt. % SiO₂. Accordingly, we concentrated on ACPS compounds that had less than 50 wt. % SiO₂; only spectra collected using these compounds were deconvuluted. Referring now to FIG. 22, the dashed lines were included to emphasize that there are three distinct regions arising from the inclusion of SiO₂ in the alloy. The upper panel displays the results from the P—O—P bending and stretching modes of the P₂O₅ network. In both cases these modes shift to higher energies when SiO₂ is added at a level of about between 5 and about 16 wt. %. Beyond this point, the modes begin to relax to lower frequencies. Physically, this trend may be due to the level of strain experienced by the P₂O₅ network. At a SiO₂ content less than 10 wt. %, the P—O—P vibrations are likely unaltered, suggesting silica is not present in sufficient quantity to dramatically affect the phosphate network. At higher quantity, up to about 16 wt. %, the silica particles occupy more volume and are forced between, in and around the P₂O₅ network due to the MC process. This induces significant strain on the P—O—P bending and stretching modes leading to a stiffening of the P₂O₅ matrix. At higher SiO₂ content some P—O—P bonds within the matrix break to alleviate strain, enabling P—O—P bonds that are still intact within the matrix, to vibrate more freely. It is possible that the stretching mode breaks earlier than the bending mode, suggesting that both the out-of-plane and in-plane bending motion offers more flexibility to accommodate strain in the material than does in-plane stretching motion.

Referring now to FIG. 22, the middle panel unexpectedly reveals an opposite trend relative to the trend observed in the upper panel. The trend illustrated in the middle panel may reflect the relaxing response of P═O and P—O⁻ vibrations to an increase in SiO₂ content. This can be explained if we consider the P₂O₅ matrix illustrated in FIG. 20. Initially silica may attach to surface groups such as the P═O and P—O⁻ groups along the P₂O₅ structure. These structures are most susceptible to structural modification and therefore precede P—O—P fracturing. At silica contents greater than 16 wt. %, however, the P—O⁻ vibration stiffens, due perhaps to cationic (e.g. Ca²⁺) attachment. This effect may become more pronounced because of an increase in the production of P—O⁻ groups due to P₂O₅ fracturing. On the other hand, at the same composition the P═O vibration flexes along with P—O—P bending and is likely mirroring the accommodation of SiO₂ particles discussed above.

Referring again to FIG. 22, (lower panel), the ionic form of PO₄³⁻ appears to stiffen then relax with increasing SiO₂ content. Presumably, the ionic form of phosphate bonds to cationic species when silica particles have yet to penetrate and significantly alter P₂O₅ arrangements by creating additional negatively-charged binding sites; when silica content is low (<10 wt. %), anionic environments are dominated by the non-network PO₄³⁻ group. When silica particles sufficiently penetrate the P₂O₅ network, the number of vacant negatively-charged sites shifts from non-network PO₄³⁻ sites to network-modified P—O⁻ sites. This effect may occur through the simultaneous loosening of P—O—P vibrations and stiffening of ionic P—O⁻ vibrations.

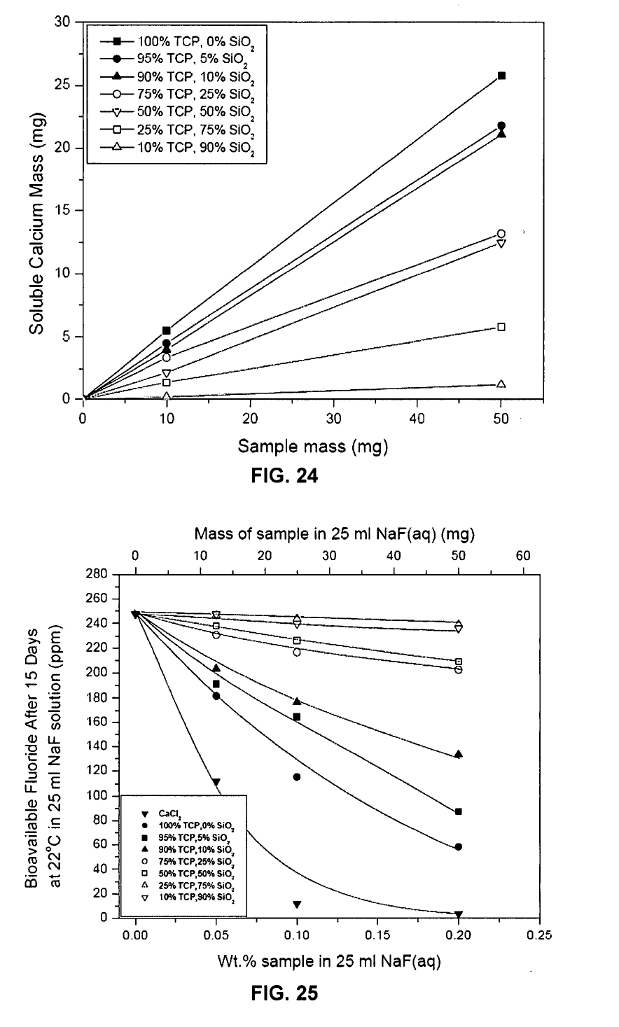

The nature of the cationic species may be explained by viewing the sharp spectral features illustrated in FIG. 19 with the P₂O₅ network illustrated in FIG. 24. A proposed mechanism of P₂O₅ network modification is described in light of FIG. 23 (Schematic 1.1) is as follows. In this schematic, calcium entities introduce and coordinate to non-bridging (i.e. non-covalent bonding) oxygen ions from the P₂O₅ network. The IR spectra illustrated in FIG. 19 indicates that spectral features near 635 cm⁻¹ are not due to P—O bonding but rather to aggregates of OCaO and CaO(O₂). The calcium aggregates observed in the material appear to form during the MC process which is carried out in this instance under ambient conditions. Somewhat unexpectedly, the energies generated during the milling process are strong enough to overcome activation energy barriers enabling these alloys to form. Besides the presence of the sharp spectral features in FIG. 19, only the intensity of the features diminishes as the content of SiO₂ increases, suggesting that less calcium is available for CaO conversion. Apparently, incorporation of calcium into the P₂O₅ network, which is meshed within the matrix, due to the flux of silica particles vying for space within the network, modifies the network straining P—O—P vibrations and creates non-bridging oxygen sites. Originally confined to the surface, matrix distortions are ultimately spread throughout the material as the silica content of the material is increased. It is the transition from a surface-modified P₂O₅ network to a bulk-modified structure that appears to characterize ACPS.

Analysis by IR spectroscopy illustrates that an alloyed calcium-phosphate-silica (ACPS) system was created using an MC milling procedure. The integrity of the microstructure may be linked to the content of SiO₂ and to the extent of network modification by unique calcium oxide moieties. The calcium that is embedded, but not covalently bonded, into the network may contribute to the coupling of fluoride and this alloy. This interaction may enabling these species to coexist in a single solution and manufacture the enhanced tissue remineralization potential observed with mixture of ACPS and fluoride relative to fluoride alone.

### Example 9

In order to further assess the stability of mixture of ACPS and fluoride we studied the effect to adding the ACPS to systems that included fluoride, Aquafresh Extreme Clean™ and Aquafresh Cavity Protection™ both of which are trademarks of GalaxoSmithKline. The results of these tests are as follows: Soluble Calcium in Various Compositions of ACPS

Referring now to FIG. 24, briefly, 0, 10, and 50 mg of each ACPS composition and controls (as shown in the legend of FIG. 24), were immersed in 100 ml of distilled water. Later, portions of each sample were tested to determine the level of water soluble calcium in each preparation. The slurries were allowed to stand for about 4 hours, at which point 1 ml aliquots were extracted and analyzed for calcium using an atomic absorption spectrometer. The results of this experiment are summarized in FIG. 24 as a series of isotherms corresponding to each ACPS composition. Surprisingly, a significant decrease in soluble calcium is observed between ACPS compositions comprising 90 and 75 wt. % SiO₂ are added relative to ACPS with lower SiO₂ content. The drop is not linear over the range tested, as illustrated by comparing values measured for materials comprising between 75 and 50 wt. % SiO₂. The aforementioned results suggest that the level of reduced calcium is not simply related to fewer calcium ions due solely to an increase in silica content necessary to maintain 100 wt. % of the material. But rather to the formation of a distinct material compound that incorporates both calcium and silica.

Referring still to FIG. 24, solubility isotherms were constructed for sample masses between 0 and 50 mg, these data were fit to a line indicating that for a given alloy of tricalcium phosphate and SiO₂ the relationship between the amount of sample tested and the amount of water soluble calcium in the sample is essentially linear. These linear fits allowed us to make reasonable estimates as to the level of soluble calcium available in the materials tested. As discussed previously, the incorporation of calcium within the P₂O₅ network may be contributing to the diminished level of soluble calcium observed with ACPS that have a high content of silica.

### Stability of Bioavailable Fluoride Measured in the Presence of ACPS and in the Absence of Added Surfactants

Referring now to FIG. 25, briefly, the thermodynamic stability of systems that included both NaF_{(aq)} and ACPS was assessed by measuring the level of bioavailable fluoride in each composition. As illustrated by the legend in FIG. 25, systems tested in this example included CaCl₂ ACP, and ACPS compositions ranging from 5 to 90 wt. % SiO₂. Samples were collected from each composition after allowing them to rest at 22° C. for 15, days all of these systems were free of added surfactants. Referring still to FIG. 25, the isotherms show that the free-form CaCl₂ apparently binds with fluoride, and reduces the level of bioavailable fluoride in the system. In contrast, the ACPS material is clearly more compatible with free fluoride than is CaCl₂. In fact, less than 15 ppm of fluoride is bound at 0.05 wt. % (500 ppm) in the ACPS alloys that included 25% or more SiO₂. This reflects a loss of only about 6% bioavailable fluoride and compares exceptionally well to the CaCl₂ system (loss of about 55% fluoride) at the same weight percent. Thus, it appears that calcium coordination within the P₂O₅ matrix, as discussed previously, prevents strong interactions with fluoride within the 15 day period. At high levels of SiO₂ content, the system models the stable silica-NaF system and the relatively low levels of calcium in these ACPS systems do not appear to contribute significantly to a reduction in bioavailable fluoride.

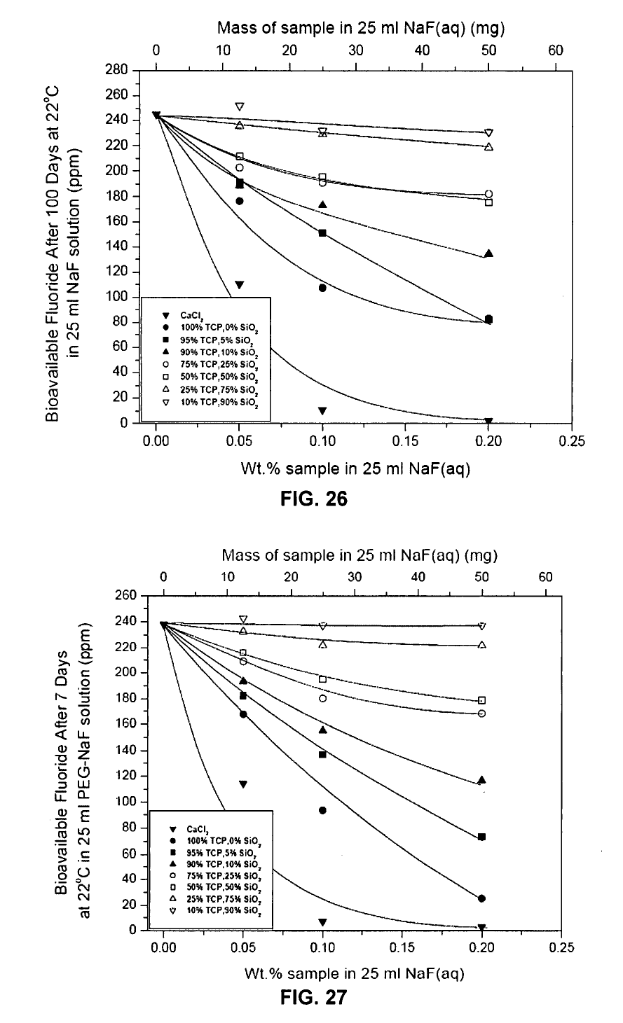

The stability of the surfactant-free NaF_{(aq)} system can be deduced by comparing the data summarized in FIGS. 25 and 26. Referring now to FIG. 26, once created each system tested was allowed to stand for 100 days prior to collecting samples and analyzing the sample to determine their levels of bioavailable fluoride (see legend of FIG. 21 for a list of materials tested). Over time, ACPS systems with 25% or more SiO₂ eventually bind with fluoride, thereby reducing the level of bioavailable fluoride in the mixture; this may be due to the loss of coordinated calcium from within the bulk P₂O₅ matrix through salvation which then becomes available to bind to fluoride in the mixture. This leads us to the conclusion that if ACPS fluoride systems are left standing in a highly solvated environment for extended periods of time (e.g. 100 days), much of the coordinated calcium in the system may be flushed from the P₂O₅ matrix, whereupon CaF₂ aggregates will form.

### Stability of Bioavailable Fluoride Measured in the Presence of ACPS and the Presence of Neutral Surfactant

Referring now to FIG. 27, formulations were prepared as follows: 600 Da polyethelyn glycol (PEG), I.O ut. % PEG was added to each formulation along with 25 of NaF_{(aq)}. Specific formulation included one of the following compounds: LaCl₂, and TCP alloyed with one of the following propositions of SiO₂, (0.0, 5, 10, 25.50, 75, or 90 nt. %) respectively (see legend of FIG. 27). Each compound was tested at three different levels: 0.05, 0.1, and 0.2 wt. % of alloy per 25 ml. of NaF_{(aq)}.

The formulation was held at room temperature, sampled after 7 days and tested for bioavailable fluoride as outlined in the above. Referring still to FIG. 27, again, the system that included CaCl₂, exhibited the strongest tendency to bind to fluoride, resulting in a reduction of about 52% at 500 ppm (0.05 wt. %) of bioavailable fluoride in the system. For systems that included ACPS, the importance of silica to the level of bioavailable fluoride in the systems is clearly evident. For example, comparing the isotherms among the ACPS to one another, ACPS alloys that included 25 wt. % or greater levels of SiO₂, (up to 0.2 wt. %, where the soluble calcium levels range between approximately 80-600 ppm), fluoride binding levels off at 25 wt. % SiO₂). These results are similar to those reported above for ACPS in the absence of surfactants over a time frame of up to 15 days. The ACPS-NaF(aq) systems manufactures charged species, and is therefore likely to be very responsive to the introduction of additional charged species into the system. In this environment, the neutral surfactant PEG does not appear to significantly affect fluoride stability.

### Stability of Bioavailable Fluoride Measured in the Presence of ACPS Added Anionic Surfactant

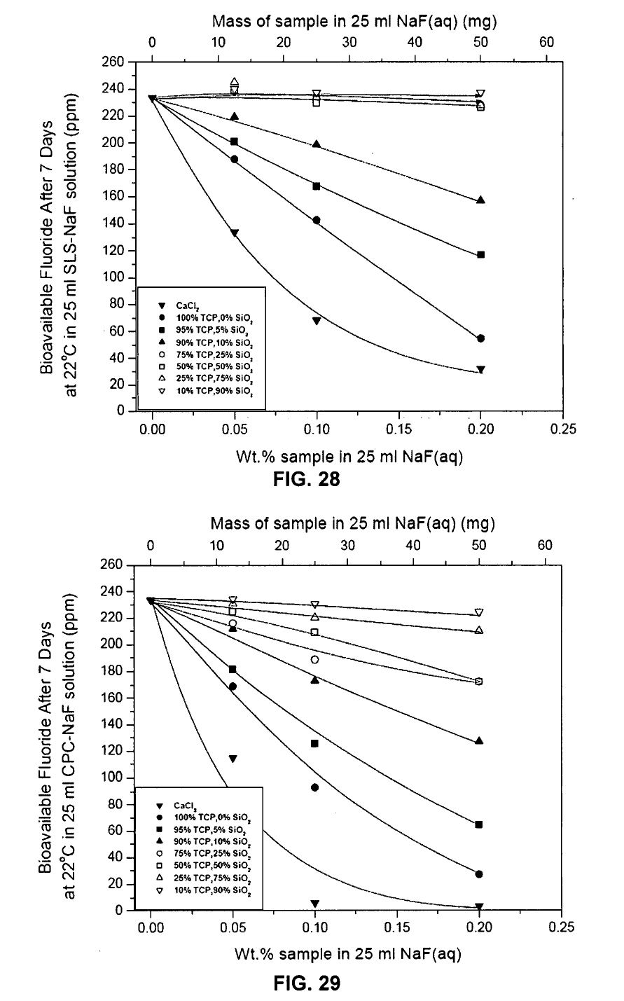

Referring now to FIG. 28, essentially the same experiment was carried out as described in FIG. 27. Referring again to FIG. 28, in this experiment PEG was replaced with the anionic surfactant 5 wt. % sodium lauryl sulfate (SLS).

The corresponding isotherms for these systems are shown in FIG. 28. A direct comparison of FIG. 28 with FIG. 27 reveals that relatively higher levels of fluoride bioavailability are retained in the presence of SLS (FIG. 28) than in the absence of the anionic surfactant (FIG. 27). The ‘best’ ACPS alloys were those providing minimum F⁻ binding within the experimental range studied (i.e. between 500 and 2000 ppm ACPS sample), and those having 25 or more weight percent SiO₂. A plausible explanation for this is that the negative sulfate group attracts soluble cations (e.g. Ca²⁺) which in turn creates ionic competition and limits reaction with F⁻. Referring to FIG. 28, further support for this hypothesis can be found in the isotherm for CaCl₂ which shows a small but significant, improvement in fluoride bioavailability in the presence of the anionic surfactant (FIG. 28) than in the absence of the surfactant (FIG. 27). These results suggest that the stability of bioavailable fluoride in the presence of ACPS can be improved by the addition of at least one anionic surfactant to the system.

### Stability of Bioavailable Fluoride Measured in the Presence of ACPS Added Cationic Surfactant

Referring now to FIG. 29 essentially the same experiment was carried out as described in FIG. 27. Referring again to FIG. 29, in this experiment PEG was replaced with the cationic surfactant 5 wt. % of cetylphridium chloride (CPC).

The results observed with the cationic surfactant (FIG. 29) are distinctly different from those observed with the anionic surfactant (FIG. 28). Referring now to FIG. 29, it appears that the quaternary ammonium species of the CPC surfactant does not screen Ca²⁺ species from F⁻ as effectively as SLS, despite the presence of anionic sites within the P₂O₅ framework as previously described.

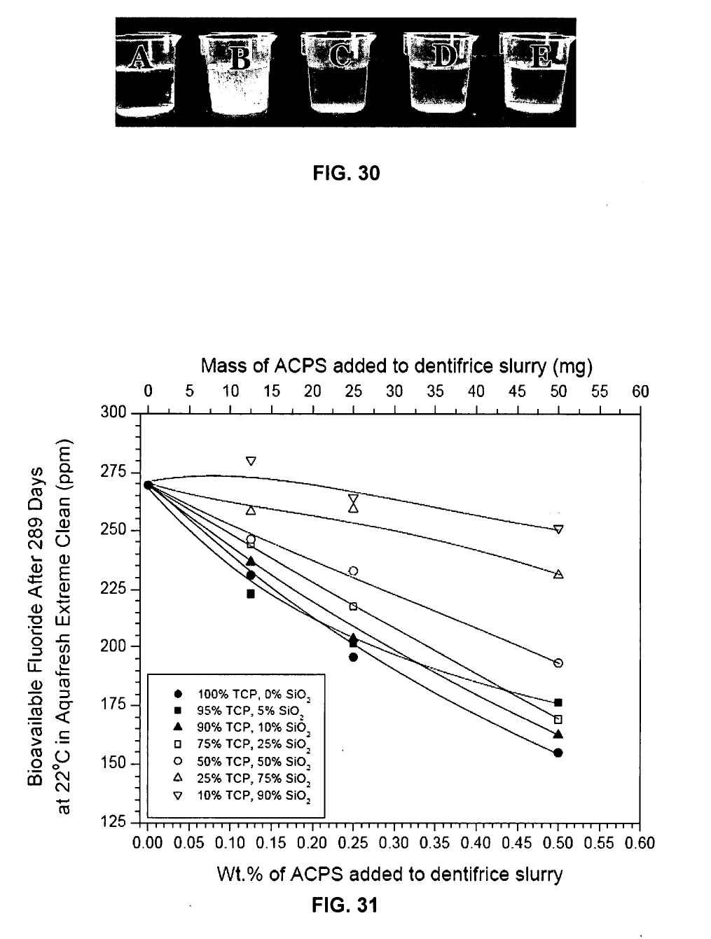

Referring now to FIG. 30, still another surprising observation is that some of the ACPS-NaF_{(aq)} systems produced a color change when CPC was added. Solutions A, B, and E remain transparent, while C and D reveal a color change. One interpretation of the results illustrated in FIG. 30 is that calcium, sodium, and fluoride ions do not respond to CPC to effectuate the color change. Instead, the color change most likely arises from the presence of charged P—O species, such as ionic PO₄³⁻ and P—O⁻ from within the covalent P₂O₅ network (e.g. FIGS. 20 and 23). Referring again to FIG. 30, the color intensity grows weaker with increasing silica content in the ACPS system, suggesting that the binding between CPC and ACPS may be influenced by a surface phenomenon, because as the extent of calcium penetration increases from the surface to within the bulk P₂O₅ network, there are fewer remaining charged P—O⁻ bonding environments to which the ammonium group of the CPC can attach. Accordingly, it appears that higher silica content in ACPS may favor the coordination of Ca²⁺ to P₂O₅ over PO₄³⁻.

### Example 10

### Stability of Bioavailable Fluoride in Various Mixtures including ACPS and Aguafresh Extreme Clean™

The effect of ACPS on the level of bioavailable fluoride was measured on mixtures of various controls and ACPS alloys which included varying amounts of SiO₂ (for a complete list of the materials tested please see the legend of FIG. 31). Briefly, the isotherms illustrated in FIG. 31 were generated by making slurries comprised of 10 grams Aquafresh Extreme Clean™, 20 ml distilled water, and the various controls and alloys shown in the legend of FIG. 31. The mixtures were sampled and analyzed for bioavailable fluoride at the end of 289 day period. Referring still to FIG. 31, the beneficial effect of silica in the ACPS alloy's ability to stabilize the level of bioavailable fluoride is obvious. Higher levels of bioavailable fluoride were measured in samples drawn from systems which included ACPS alloys that had at least 25 wt. % SiO₂.

Due to the complicated formulation of this commercially available dentifrice (Aquafresh Extreme Clean™) it is difficult to assess the nature of the interactions arising between fluoride in the dentifrice and ACPS. Based on the stability studies with various surfactants discussed above, the Extreme Clean system may be contributing surfactants or similar agents that bind fluoride to surface sites of the P₂O₅ network. This effect would be more pronounced at longer times due to fluoride and/or calcium diffusion into and out of the P₂O₅ network. In any event ACPS alloys that are comprised of at least 25 wt. % SiO₂ yield surprisingly high levels of bioavailable fluoride when added to this commercially available dentifrice (i.e. a loss of less than 10% bioavailable fluoride). Accordingly, the addition of the ACPS to the Extreme Clean formulation will likely provide improved remineralization when tested in vitro dental testing models.

### Example 11

### Stability of Bioavailable Fluoride in Various Mixtures including ACPS and Aguafresh Cavity Protection™

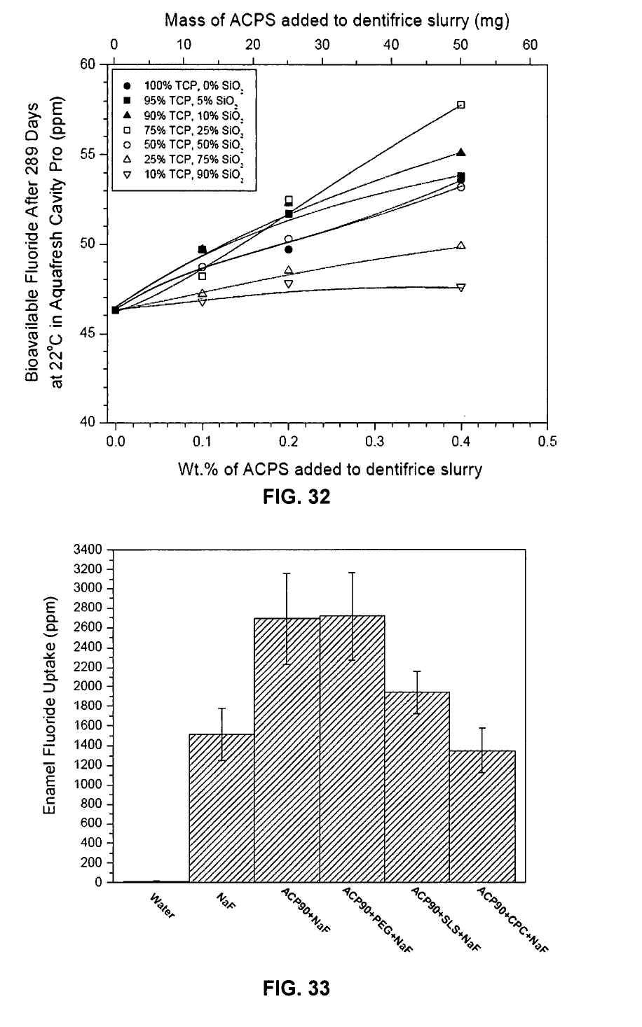

Referring now to FIG. 32, the effect of ACPS on the level of bioavailable fluoride was measured for mixtures of Aquafresh Cavity Protection™ and various ACPS alloys which included varying amounts of SiO₂ (for a comprehensive list of the materials tested please see legend of FIG. 32). Briefly, 12.5 grams of Cavity Protection paste, 25 ml distilled water, and 0.1, 0.2, or 0.4 wt. % ACPS, respectively were mixed to form 6 different systems. The mixtures were sampled after 289 days and assayed to determine the level of bioavailable fluoride in each system. The resulting values were plotted as a function of the wt. % of ACPS added to each system. The data were fit to lines as illustrated in FIG. 32. The isotherms in FIG. 32 illustrate ACPS alloys are not only compatible with NaMFP in the Cavity Protection formulation but also enhance the level of bioavailable material in dentifrice.

Surprisingly, ACPS alloys, relatively low in silica appear to generate the best improvements in bioavailable fluoride when added to the system in low amounts (i.e. 0.05 wt. %). When ACPS was added at higher levels (>0.1 wt. %), all ACPS alloys at this level increased the level of bioavailable fluoride. The greatest increase in bioavailable fluoride was observed with ACPS alloys having a silica content of less than 50 wt. % SiO₂. We note that Cavity Protection™ is formulated with calcium carbonate and ACPS alloys are thought to have surface-active calcium ions. Accordingly, the improvement in fluoride release observed by adding ACPS may be due to interactions among the MFP species and charged surface P₂O₅ sites of the ACPS alloys. These results indicate that the addition of ACPS to this dentifrice formulation is likely to promote enhanced remineralization of enamel lesions.

In these studies, anionic surfactants such as SLS appeared to provide the best stability with NaF when combined with ACPS. Preparations without surfactants, or those with neutral or positive surfactants, exhibited lower levels of bioavailable fluoride than systems which included ACPS and the anionic surfactant (SLS). Furthermore, the ACPS alloys tested herein were shown to be surprisingly compatible with existing commercially available dentifrices formulated with fluoride. Indeed in these studies the addition of ACPS alloys to mixtures of the two dentifrices tested increased the level of bioavailable fluoride from NaMFP, when measured after 289 days of contact between ACPS and NaMFP.

### Example 12

### Testing the Effect of Alloys of Amorphous Tricalcium Phosphate and Metal Oxide on Enamel Fluoride Uptake (EFU)

Briefly, sound, upper, central, and bovine incisors were selected and cleaned of all adhering soft tissue. A core of enamel 3 mm in diameter is prepared from each tooth by cutting perpendicularly to the labial surface with a hollow-core diamond drill bit. The operation was performed under water to prevent overheating of the specimens. Each specimen was embedded in the end of a plexiglass rod (¼″ diameter×2″ long) using methylmethacrylate. The excess acrylic was cut away to expose the enamel surface. The enamel specimens were polished with 600 grit wet/dry paper and then with micro-fine Gamma Alumina. The resulting specimen was a 3 mm disk of enamel with all but the exposed surface covered with acrylic.

Each enamel specimen was then etched by immersion into 0.5 ml of 1 M HClO₄ for 15 seconds. The etching solutions were agitated continuously throughout the etching period. A sample of each solution was then drawn and buffered with TISAB to a pH of 5.2 (0.25 ml sample, 0.5 ml TISAB and 0.25 ml 1N NaOH) and the fluoride content of each sample was determined by comparison to a similarly prepared standard curve (1 ml std+1 ml TISAB). To establish a baseline for interpreting these results data were collected to determine the indigenous fluoride level of each specimen prior to treatment.

The specimens were once again ground and polished as described above. An incipient lesion is formed in each enamel specimen by immersion into a 0.1M lactic acid/0.2% Carbopol 907 solution for 24 hours at room temperature. The specimens were then rinsed well with distilled water and stored in a humid environment until they were used. The treatments were performed using supernatants of the dentifrice slurries, comprising 1 part dentifrice and 3 parts distilled water (9 g:27 ml). The specimens were then immersed into 25 ml of their assigned supernatant with constant stirring (350 rpm) for 30 minutes. Following treatment, the specimens were rinsed with distilled water. One layer of enamel was then removed from each specimen and analyzed for fluoride as outlined above. The pretreatment fluoride (indigenous) level of each specimen was then subtracted from post treatment value to determine the change in enamel fluoride content due to each treatment. Statistical differences were assessed by evaluation of individual means using ANOVA. When the data indicated that there were significant differences among the means (p<0.05), the means were further analyzed using either multiple t-tests or the SNK test.

The test groups examined in this EFU study are as follows: 1) Water (negative control); 2) 250 ppm F⁻ solution (positive control); 3) ACP+CPC+250 ppm F⁻ solution; 3) ACP50+CPC+250 ppm F⁻ solution; 4) ACP50+SLS+250 ppm F⁻ solution; 5) ACP90+PEG+250 ppm F⁻ solution; 6) ACP90+CPC+250 ppm F⁻ solution; 7) ACP90+SLS+250 ppm F⁻ solution; and 8) ACP90+250 ppm F⁻ solution. Approximately 12.5 mg of the ACPS powder (either ACP, ACP90, or ACP50) is added to 25 ml of 250 F⁻ solution (prepared by dissolving NaF into DI water) to give an ACPS concentration of 500 ppm.

Referring now to FIG. 33. FIG. 33 summarizes experiments carried out to assess the efficacy of Enamel Fluoride Uptake (EFU)I in systems that included the following ACPS alloys: 90% TCP, 10% SiO₂. Values were measured in the presence and absence of various surfactants (see legend of FIG. 33). As the data presented in FIG. 33 illustrate, ACPS comprising 10 wt. % SiO₂, performs surprisingly well in terms of promoting fluoride update by enamel in the system. This result was under all experimental conditions tested i.e. with no added surfactant as well as with either the neutral (polyethylene glycol, PEG), or anionic (SLS) surfactants. The presence of ACPS incorporating 10 wt. % SiO₂ appears to screen at least a portion of the calcium-modified P₂O₅ network of ACPS from interacting with fluoride. On the other hand, preparations that included the cationic surfactant (cetylpyridinium chloride, CPC) did not perform as well as those preparations that included no surfactants or neutral or anionic surfactants. The cationic surfactant CPC may encourage electrostatic interactions among the quaternary ammonium ion, the charged surface groups of the Ca²⁺-doped P₂O₅ network of ACPS, and bioavailable fluoride. It is possible that CPC does not shield Ca²⁺ from F⁻ as effectively as does SLS; therefore, at least a fraction of the available fluoride may become bound to ACPS effectively reducing the level of bioavailable fluoride available for uptake into the enamel.

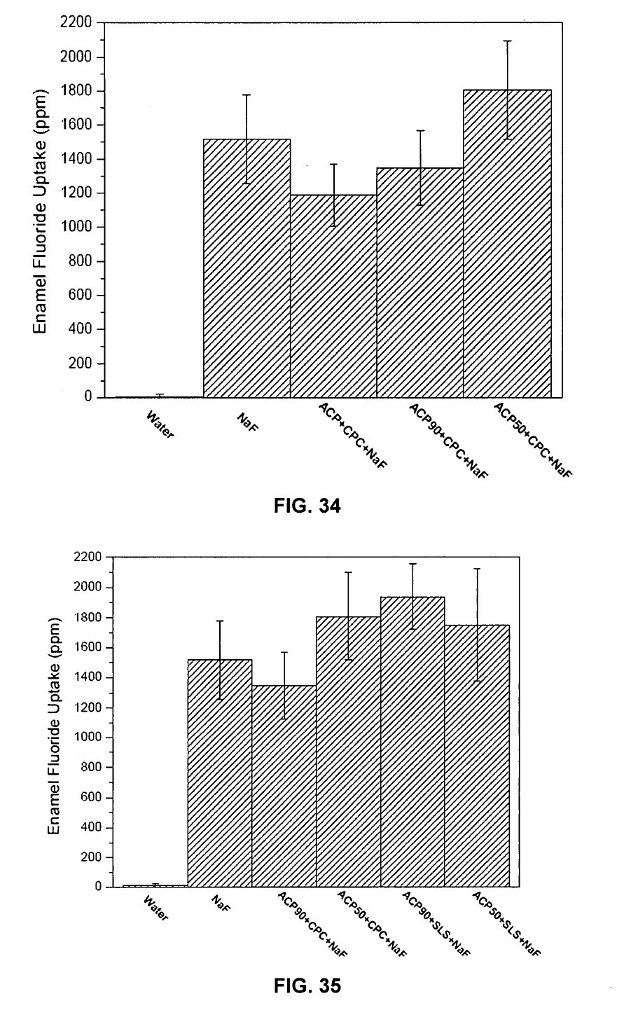

Referring now to FIG. 34, the effect of ACPS alloy preparations comprising, 0, 10 and 50 wt. % SiO₂ on enamel fluoride uptake was determined and reported herein in the form a series of histograms. Systems that included alloys of ACPS incorporating 50 wt. % SiO₂ exhibited higher levels of Enamel Fluoride Uptake relative to systems that included ACPS incorporating either no or 10 wt. % SiO₂ in systems that also included the cationic surfactant CPC. Apparently, ACPS alloys with higher SiO₂ content better shielded Ca²⁺ atoms in the alloys from binding fluoride thereby leaving more bioavailable fluoride in the system for incorporation in the enamel surface.

Referring now to FIG. 35, the effect of ACPS including different levels of SiO2 (either 10 or 50 wt. %) was measured in the presence or absence of different surfactants. As the histograms in FIG. 35 illustrate, preparations which included ACP alloyed with 50 wt. % SiO2 showed virtually no difference when either the anionic (SLS) or cationic surfactants (CPC) were added to the system. In this system, calcium incorporation into sites of the P2O5 network appears to be extended from the surface to the bulk material. This is consistent with the microstructure infra-red analysis reported earlier as well as visible color changes of solutions comprised of ACPS and CPC also reported earlier. On the other hand, the ACP90 system is clearly influenced by SLS and CPC as described above. Accordingly, the level of metal oxide such as SiO₂ alloyed to amorphous tricalcium phosphate has an effect on the systems ability to promote EFU. Also, as illustrated by the data presented herein, it is possible to effect EFU by the addition of surfactants to systems that include alloys comprised of amorphous tricalcium phosphate and metal oxides alloys and fluoride.

### Example 13

### Resistance of Enamel Surfaces to Acid Etching Measured by Observing Enamel Etching Depth

We also investigated differences in the depth of enamel etching in the presence of ACPS, with and without various surfactants. The same solutions and materials used in example 12, were used here in example 13. Essentially, the only difference is that in example 13 we measured the change in the depth enamel etching rather than the level of fluoride uptake.

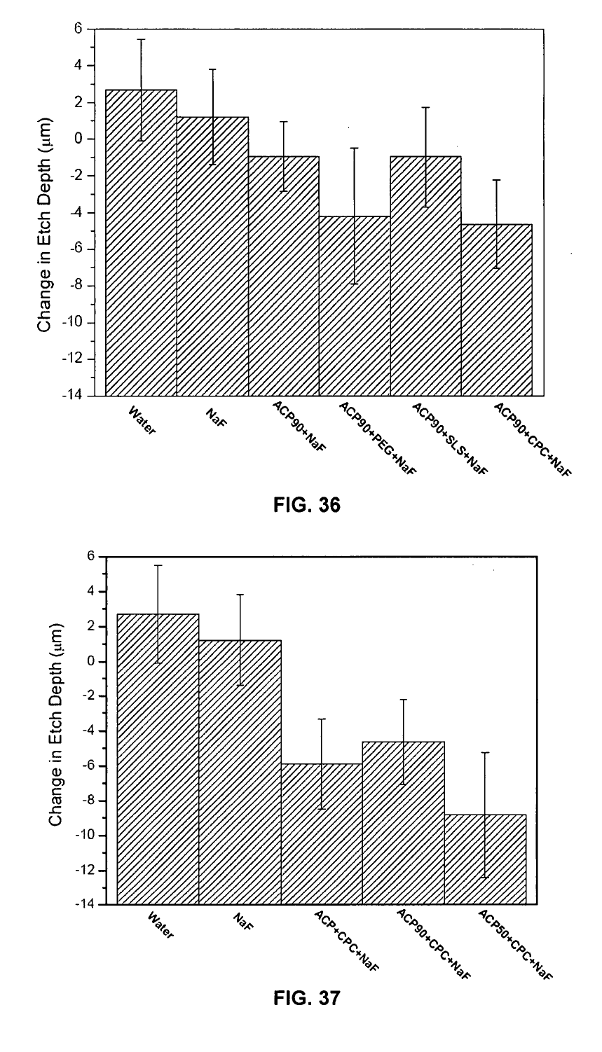

Briefly, enamel surfaces were exposed to percchoric acid which etches enamel. The depth of the enamel surface was measured before and after the exposure and the results are presented in the figures as Change in Etch Depth (CED). In these figures, conditions with larger negative CED values are indicative of conditions that better protect the enamel surface from acid etching. FIGS. 36-38 summarize results collected using 3 types of surfactants and preparations of ACPS including no added SiO₂ (ACP) and either 10 (ACP₉₀) or 50 (ACP₅₀) wt. % SiO₂.

Referring now to FIG. 36, it is evident from these histograms that the incorporation of the nano-sized ACP metal oxide alloy assists in preventing enamel dissolution. This may be due to the formation of fluorapatite or another form of mineral comprised of calcium, phosphorous, and fluoride that is less soluble than hydroxyapatite.

Referring now to FIG. 37, when CPC was added to the systems, the best results (lowest level of enamel etching) was observed when ACPS incorporating 50 wt. % SiO₂ was added to the system. One plausible explanation for these results is that CPC does not effectively shield the ACPS or ACP from fluoride. Accordingly, F⁻ can interact with Ca²⁺ in the ACP and ACP90 (10 wt. % SiO₂) systems and it is possible that this interaction may produce small clusters that precipitate mineral phases near the enamel surface. On the other hand, the ACP50 system may have limited F⁻ interactions (based on F⁻ stability studies as previously discussed). The net result is that there is more bioavailable fluoride when the system includes ACP50 (50 wt. % SiO₂). It may be that this alloy is interacting with enamel in a synergistic way to extend mineral uptake deeper into enamel. This explanation can be inferred from the greater protection afforded by this formulation relative to formulation that included either ACP and or ACP90 (10 m wt. % SiO₂).

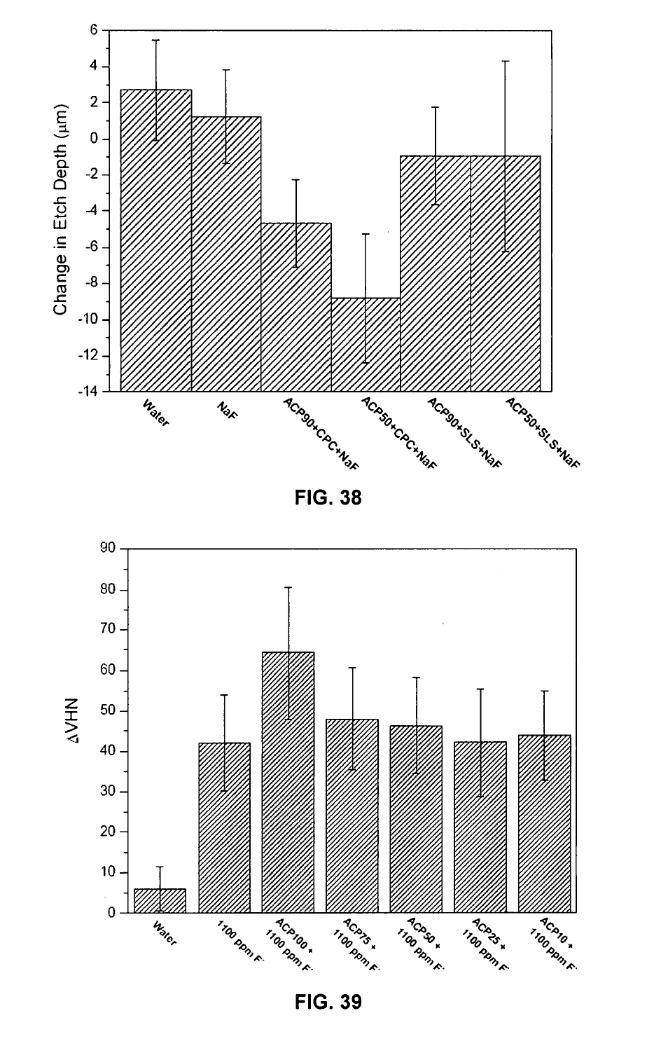

Referring now to FIG. 38, the best results (least enamel etching) was observed with ACP50 (50 wt. % SiO₂), CPC and NaF. Unexpectedly, enamel surfaces treated with ACP90 (10 wt. % SiO₂), SLS and NaF, exhibited more etching than the surfaces treated with the alloy and the cationic surfactant CPC. This may be due to the formation of calcium fluoride precipitates that from in the presence of CPC and fill in imperfections in the enamel surface.

### Example 14

### Remineralization/Demineralization pH Cycling Using Alloys of Amorphous Tricalcium Phosphate and Metal Oxides

A remineralization/demineralization pH cycling study was carried out as follows. As there is little difference in mineral matrix and lesion formation between bovine and human enamel, either type of enamel specimens (5 mm×5 mm) were extracted from teeth and mounted in rods as outline above. The specimens were ground and polished to a high luster with Gamma Alumina (0.050 microns) using standard methods. Ten specimens per group were prepared for each study. Artificial lesions were formed through immersion of specimens in a solution comprising 0.1 M lactic acid and 0.2% Carbopol C907. This solution is 50% saturated with hydroxyapatite and adjusted to a pH of 5.0. The baseline lesion surface microhardness range spanned 20 to 50 Vickers hardness numbers and average lesion depth was approximately 70 microns.

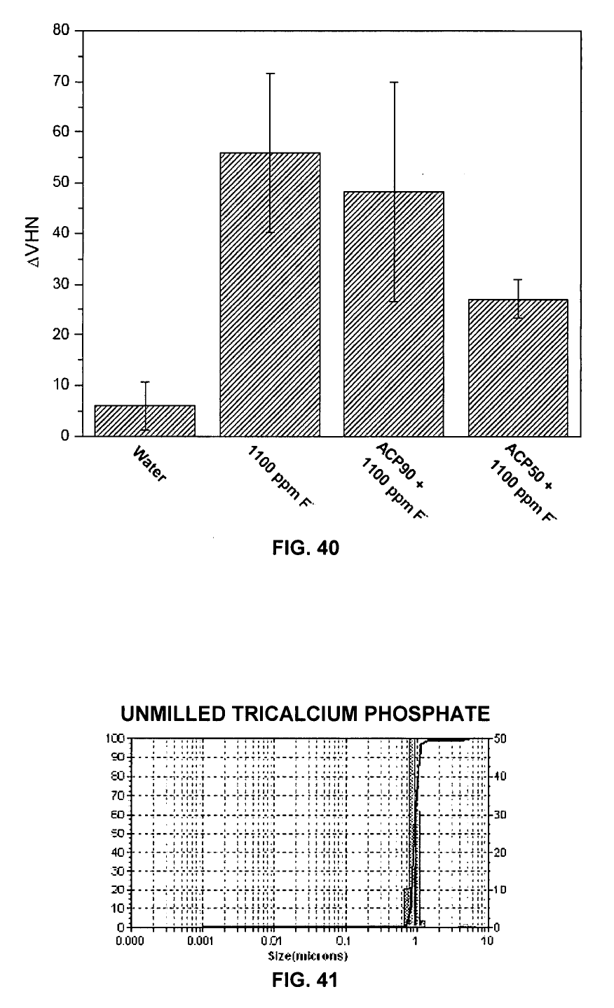

The treatment regimen consisted of 1, four-hour/day acid challenges in the lesion forming solution, and 4, two-minute treatments each day in a slurry. The composition of the slurries differed. For a summary of the composition used in this study see the legends of the corresponding FIGS. 39 and 40. After each slurry treatment at the completion of each cycle, the specimens were placed in an artificial mineral mix as described by Cate, et al. This process was repeated for 6 or 10 days. A typical schedule used in this cycling model is: a. 8:00-8:02 a.m.—Dentifrice treatment*; b. 8:02-9:00 a.m.—Saliva treatment; c. 9:00-9:02 a.m.—Dentifrice treatment; d. 9:02-10:00 a.m.—Saliva treatment; e. 10:00 a.m.-2:00 p.m.—Acid challenge; f. 2:00-3:00 p.m.—Saliva treatment^{‡}; g. 3:00-3:02 p.m.—Dentifrice treatment; h. 3:02-4:00 p.m.—Saliva treatment; i. 4:00-4:02 p.m.—Dentifrice treatment; j. 4:02-overnight.—Saliva treatment; and k. Back to (a). On days labeled by an asterisk (*) the specimens were immersed in artificial saliva to establish the pellicle. On days labeled by a double dagger (^{‡}) fresh saliva changed.

Remineralization efficacy was judged by comparing post Vickers microhardness numbers to baseline Vickers values. Means and standard deviations of the means were calculated and the Student Q-test was used to assess accuracy of the individual specimen measurements within each group. Statistical analysis was performed using the Kruskal-Wallis one-way analysis of variance on ranks (ANOVA) to test for the presence of significant differences (p<0.05). If significant differences were found to exist, multiple comparisons of the individual means were analyzed with multiple t-tests (e.g. Dunn's or SNK method).

The first study spanned many compositions of the ACPS system that were added to 1100 ppm F⁻ solution (i.e. NaF_{(aq)}), including 0, 25, 50, 75, and 10 wt. % SiO₂. In this particular study, 5×5 mm bovine enamel specimens were used and fresh treatment solutions were prepared by adding 0.15 wt. % ACPS powder to 5 ml NaF_{(aq)} and 10 ml artificial saliva and mixing them no more than 2 minutes prior to treatment of specimens. Each treatment lasted two minutes and after six days of cycling, the change in Vickers microhardness was determined as shown in FIG. 39. All test groups broke statistically from the negative control, however, only the ACP100 system broke statistically from the positive control.

The second study examined only two ACPS systems (ACP90 and ACP50) when they were mixed with 1100 ppm F⁻ solutions (i.e. NaF_{(aq)}). In this particular study, 3 mm human enamel specimens were used and fresh treatment solutions were prepared at least one hour prior to treatment by adding 0.15 wt. % ACPS powder to 5 ml NaF(aq) to allow for phase mixing (solutions were not mixed, however). Then, prior to treatment, 10 ml artificial saliva was added to the prepared slurry mixed for less than 2 minutes prior to treatment of specimens. Each treatment lasted two minutes and after ten days of cycling, the change in Vickers microhardness was determined the results are presented in FIG. 39. The positive control and the ACP90 test group broke statistically from the negative control, while the ACP50 system did not. Numerically, the positive control performed better than the two ACPS groups, however there was no statistical difference between the positive control and the ACP90 test group. It appears as though that the color of the enamel specimens indicates remineralization, in that brown enamel specimens produced smaller Vickers indentations (and are therefore harder) than specimens with little or no brown at all (i.e. those that are colorless). In this study, the group most white was the negative control. A clear trend was observed in the amount of brown, with the positive control exhibiting the most. Evidently, this coloring can be attributed to the incorporation of fluoride into the enamel lesion to promote fluorosis and/or discolorations.

The different enamel responses between the two studies may be associated with the interaction time between the ACPS system and the NaF_{(aq)} solution and concentration of fluoride. For instance, in the EFU study specimens were immersed in agitated slurries comprising ACPS and 250 ppm F⁻ for 30 minutes. Additional fluoride stability studies were also performed in 250 ppm F⁻ solutions. In the second pH cycling study (FIG. 40), ACPS was exposed to 1100 ppm F⁻ for one hour prior to dilution with artificial saliva. It appears that F⁻ concentration affects the synergy ACPS and fluoride. The fact that enamel specimens were less brown in the ACP90 and ACP50 experimental groups in (FIG. 40) may indicate that that fluoride became bound to the nanosized silica (15 nm particles). Fewer soluble calcium ions are available in the ACP50 system (as shown in the stability studies previously discussed), therefore these compositions are less likely to bind with fluoride and reduce fluoride bioavailability. It is likely that ACPS particles used in this system affect F⁻ binding due to the inherently large surface area and partial charges of ACPS.

When surfactants are added to the formulations, the behavior and compatibility of the ACPS and fluoride systems change dramatically, as shown in the various stability isotherms of ACPS-fluoride formulations (including Aquafresh NaF and NaMFP systems), as well as in the EFU results. It is likely then the omission of surfactants (both positive and negative) in systems comprising ACPS will affect the remineralizing synergy, especially if fluoride concentrations are high (e.g. 1100 ppm F⁻), due to the absence of charged species covering the reactive silica system. For example, we found that SLS provided the best compatibility between ACPS and fluoride among charged and neutral surfactants. Presumably, the positive partial charges present in ACPS interface strongly with the sulfate groups of SLS. On the other hand, cationic surfactant could also be added simultaneously to further enamel fluoride uptake, as shown in FIG. 35, for example.

In the absence of added surfactants to help stabilize the ACPS-fluoride system, it may be possible to achieve higher fluoride concentrations, using the silica phase typically used in conventional toothpaste instead of using nanosized particles of silica. Switching to conventional silica should reduce the reactivity of the silica component and may also prove to be a more cost-effective approach as well. Additionally, substituting conventional silica for nanosized silica would not change materials processing parameters and should not significantly alter the P₂O₅ network or CaO aggregates as discussed previously. This is likely the case because Si—O covalent bonds are incredibly strong relative to Ca—O and P—O ionic bonds. Accordingly, although silica particles may become slightly reduced in size, the silica chemistry should be left intact since the energies generated in the ball mill are likely not sufficient to induce cleavage of these covalent bonds.

### Example 15

### Measurements of the Particle Size of Some Material Made in According to Some of the Embodiments

Referring now to FIGS. 41, 42 and 43. The particle size of various materials was measured using a Nanopac 151 particle scanner manufactured by Microtrac™. All samples were prepared and analyzed in accordance with the manufacturer's instructions.

Referring now to FIG. 41, unmilled tricalcium phosphate was analyzed using the Nanopac 151 as described above. Referring still to FIG. 41, the trace shows that this material has an average particle size on the order of about 1 micron.

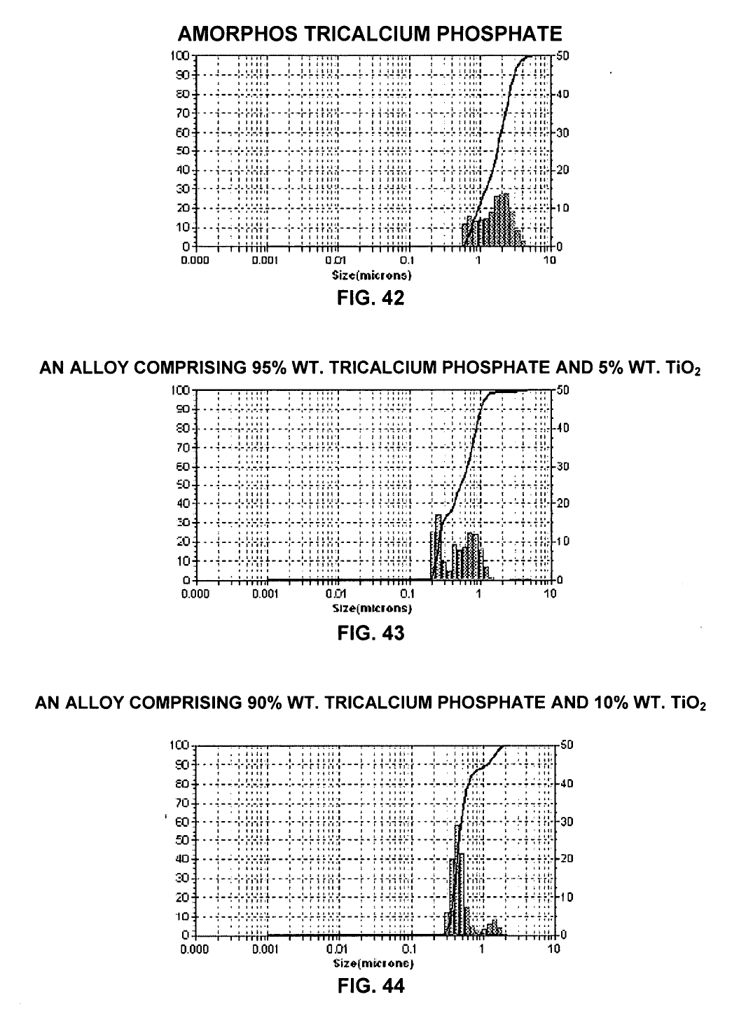

Referring now to FIG. 42, a Nanopac 151 trace showing the average particle size of a sample of amorphous tricalcium phosphate. The amorphous tricalcium phosphate material was made as follows: 20 gm of unmilled tricalcium phosphate was added to a 150 ml stainless steel vessel. The vessel also contained 20 stainless steel balls, each ball having a diameter of about 10 mm and about 5 ml of ethanol to prevent caking of the powder. The vessel was sealed, mounted on a ball mill and milled at 350 rpm for about 24 hours under ambient conditions. Once the milling was complete, the vessel was opened and the powder sampled and analyzed to determine its particle size. Referring still to FIG. 42, the average particle size of the amorphous tricalcium phosphate was on the order of about 0.8 to about 5 microns.

Referring now to FIG. 43, a Nanopac 151 trace showing the average particle size of a sample of an alloy of 95 wt. % amorphous tricalcium phosphate and 5 wt. % TiO₂. The alloy was made as follows: 20 gm total of 95 wt. % amorphous tricalcium phosphate and 5 wt. % TiO₂ was added to a 150 ml stainless steel vessel. The vessel also contained 20 stainless steel balls each ball having a diameter of about 10 mm, and about 5 ml of ethanol to prevent caking of the powder. The vessel was sealed, mounted on a ball mill and milled at 350 rpm for about 24 hours under ambient conditions. Once the milling was complete, the vessel was opened and the powder sampled and analyzed to determine its particle size. Referring still to FIG. 43, the average particle size of the alloy was on the order of about 0.2 microns to about 1.1 microns. Compared to the traces in FIGS. 41 and 42, this trace in FIG. 43 is clearly different, showing the formation of a unique compound an alloy of tricalcium phosphate and TiO₂ oxide.

Referring now to FIG. 44, a Nanopac 151 trace showing the average particle size of a sample of an alloy of 90 wt. % amorphous tricalcium phosphate and 10 wt. % TiO₂. The alloy was made as follows: 20 gm total of 95 wt. % amorphous tricalcium phosphate and 5 wt. % TiO₂ was added to a 150 ml stainless steel. The vessel also contained 20 stainless steel balls, each ball having a diameter of about 10 mm. About 5 ml of ethanol was added to prevent caking of the powder. The vessel was sealed, mounted on a ball mill and milled at 350 rpm for about 24 hours under ambient conditions. Once the milling was complete, the vessel was opened and the powder sampled and analyzed to determine its particle size. Referring still to FIG. 44 the average particle size of the alloy was on the order of about 0.6 to about 1.5 microns. Compared to the traces in FIGS. 41 and 42 this trace in FIG. 44 is clearly different showing the formation of a unique compound an alloy of tricalcium phosphate and TiO₂ oxide.

While the invention has been illustrated and described in detail in the figures and foregoing description, the same is to be considered as illustrative and not restrictive in character, it being understood that only the preferred embodiments have been shown and described and that all changes and modifications that come within the spirit of the invention are desired to be protected. As well, while the invention was illustrated using specific examples, theoretical arguments, accounts, and illustrations, these illustrations and the accompanying discussion should by no means be interpreted as limiting the invention. All patents, patent applications, journal articles, texts, treatises, publications, and the like referenced in this application are incorporated herein by reference in their entirety as if each were incorporated individually.

I claim:

1. An alloy, comprising;
amorphous tricalcium phosphate; and
at least one metal oxide.

2. The alloys according to claim 1, wherein the alloy has an average particle size in the range of about 5.0 microns to about 0.01 microns.

3. The alloys according to claim 1, wherein said alloy has an average particle size in the range of about 1.2 microns to about 0.07 microns.

4. The alloy according to claim 1, wherein said metal oxide is selected from the group consisting of: TiO₂ and SiO₂.

5. The alloy according to claim 1, wherein the amount of the amorphous tricalcium phosphate in the alloy is the range of about 99.5 to about 1.0 wt. % of the alloy and the amount of the metal oxide in the composition is in the range of about 0.5 to about 99.0% wt. % of the alloy.

6. The alloy according to claim 1, wherein the amount of the amorphous tricalcium phosphate in the alloy is the range of about 99.5 to about 85 wt. % of the alloy and the amount of the metal oxide in the composition is in the range of about 0.5 to about 15% wt. % of the alloy.

7. A method of forming an alloy, comprising the steps of:
providing a portion of amorphous tricalcium phosphate;
supplying a portion of at least one metal oxide; and
pulverizing said portion of amorphous tricalcium phosphate and said portion of at least one metal oxide together to form an alloy.

8. The method according to claim 7, wherein the alloys has an average particle size in the range of about 5.0 microns to about 0.01 microns.

9. The method according to claim 7, wherein said alloy has an average particle size in the range of about 1.2 microns to about 0.07 microns.

10. The method according to claim 7, wherein said pulverizing step is carried out in a ball mill, wherein said ball mill is operated at between about 250 to about 600 rpms, for between about 5 hours to about 5 days.

11. A formulation for treating tissue, comprising:
at least one alloy, wherein said alloy includes amorphous tricalcium phosphate and at least one metal oxide.

12. The formulation according to claim 11, wherein the alloys has an average particle size in the range of about 5.0 microns to about 0.1 microns.

13. The formulation according to claim 11, wherein said alloy has an average particle size in the range of about 1.2 microns to about 0.1 microns.

14. The formulation according to claim 11, wherein the at least one metal oxide is selected from the group consisting of: TiO₂ and SiO₂.

15. The formulation according to claim 11, wherein the amount of the amorphous tricalcium phosphate the alloy is between about 99.5 to about 1.0 wt. % of the alloy and the amount of the metal oxide in the alloy is between about 0.5 to about 99.0% wt. % of the alloy.

16. The formulation according to claim 11, wherein the amount of the amorphous tricalcium phosphate in the alloy is the range of about 99.5 to about 85 wt.

17. The formulation according to claim 11, further including at least one of the following additives selected from the group consisting of: fluoride, surfactants, antimicrobials, flavoring agents, detergents, coloring agents, buffering agents, thickening agents, cooling agents, glues, cements, and polishes.

18. A method of treating tissue, comprising the steps of:
providing a formulation that includes at least one alloy, said alloy including amorphous tricalcium phosphate and at least one metal oxide; and
contacting said preparation with at least one surface of a tissue.

19. The method according to claim 18, wherein the alloy has an average particle size in the range of about 5.0 microns to about 0.01 microns.

20. The method according to claim 18, wherein the alloy has an average particle size in the range of about 1.2 microns to about 0.07 microns.

21. The method according to claim 18, wherein the at least one metal oxide is selected from the group consisting of: SiO₂ and TiO₂.

22. The method according to claim 18, wherein the tissue is selected from the group consisting of: bone, enamel and dentin.

23. The method according to claim 18, wherein said formulation further includes at one of the compounds selected from the group consisting of: a form of bioavailable fluoride, surfactants, flavoring agents, coloring agents, thickening agents, antimicrobial agents, buffering agents, and gums.

24. A foodstuff, comprising:
an alloy, wherein said alloy includes amorphous tricalcium phosphate and at least one metal oxide.

25. The method according to claim 24, wherein the alloys has an average particle size in the range of about 5.0 microns to about 0.01 microns.

26. The method according to claim 24, wherein said alloy has an average particle size in the range of about 1.2 microns to about 0.07 microns.

27. The foodstuff according to claim 24, wherein said metal oxide is selected from the group consisting of: SiO₂ and TiO₂.

28. The foodstuff according to claim 24, wherein the amount of said amorphous tricalcium phosphate in the alloy is between about 99.5 to about 1.0 wt. % of the alloy and the amount of the metal oxide in the alloy is between about 0.5 to about 99.0 wt. % of the alloy.

29. The foodstuff according to claim 24, further including at least one compound selected from the group consisting of: a form of fluoride, a gum, a flavoring agent, a coloring agent, a cooling agent, a surfactant, a buffer, an antimicrobial agent, and a stabilizer.

30. An antimicrobial compound comprising:
amorphous tricalcium phosphate, wherein said amorphous tricalcium phosphate has an average particle size less than about 1.5 microns or less.

31. The method of manufacturing an antimicrobial composition, comprising the steps of:
providing at least one form of tricalcium phosphate;
pulverizing said at least one form of tricalcium phosphate until it is amorphous and has a particle size on the order the particle size of amorphous tricalcium phosphate manufactured by pulverizing about tricalcium phosphate in a PM 100 planetary ball mill, the mill having a stainless steel vessel with a volume of about 150 ml, said vessel including 25 stainless steel balls, wherein each of the balls has a diameter of about 10 mm, and about 2 ml of ethanol, said ball mill is operated at about 450 rpms for about 5 days.

32. The composition according to claim 31, wherein said tricalcium phosphate is formed by:
combining about equal amounts of calcium carbonate (CaCO₃) and calcium phosphate dehydrate (CaHPO₄)₂.2H₂O); and
heating said mixture to about 1050 degrees centigrade for about 24 hours.

33. A method of controlling microbes, comprising the steps of:
providing amorphous tricalcium phosphate; and
contacting said powder with at least one surface.

34. The method according to claim 22, wherein said amorphous tricalcium phosphate has a particle size on the order of the particle size of amorphous tricalcium phosphate manufactured by pulverizing tricalcium phosphate in a PM 100 planetary ball mill, the mill having a stainless steel vessel with a volume of about 150 ml, said vessel including 25 stainless steel balls, wherein each of the balls has a diameter of about 10 mm, and about 2 ml of ethanol, and said mill is operated at about 450 rpms for about 5 days.

35. The method according to claim 33, wherein said amorphous tricalcium phosphate is contacted with a device selected from the group consisting of: bandages, screws, fillings, straps, packings, nails, splints, implants, catheters, prosthetic devices, stent needles, lances, surgical tools, meshes, sutures, and endoscopes.

36. The method according to claim 33, wherein said amorphous tricalcium phosphate added to at least one of the following formulations: a dentifrice, a mouth wash, a mouth rinse, a mouth spray, a tooth whitener, a periodontal packing, a surgical packing, a foodstuff, a glue, a cement, a filling material, and a disinfectant.
